(19) United States
(12) Patent Application Publication
(10) Pub. No.: US 2024/0390895 A1
(43) Pub. Date: Nov. 28, 2024

(54) SELF-POWERED CAPILLARY MICROFLUIDIC-BASED ELECTROCHEMICAL BIOSENSING DEVICES, SYSTEMS, AND METHODS

(71) Applicant: Critical Care Dx Ltd., Calgary (CA)

(72) Inventors: Amir SANATI-NEZHAD, Calgary (CA); Razieh SALAHANDISH, Calgary (CA); Sultan KHETANI, Calgary (CA); Fatemeh HAGHAYEGH, Calgary (CA)

(21) Appl. No.: 18/696,675

(22) PCT Filed: Sep. 28, 2022
(86) PCT No.: PCT/CA2022/051439

§ 371 Date: Mar. 28, 2024

Related U.S. Application Data
(60) Provisional application No. 63/262,018, filed on Oct. 1, 2021.

(51) Int. Cl. B01L 3/00; C09D 11/106; C09D 11/52; G01N 27/327; G01N 33/53

(52) U.S. Cl. CPC B01L 3/502715; C09D 11/106; C09D 11/52; G01N 27/3278; G01N 33/5304; B01L 2200/16; B01L 2300/047; B01L 2300/0645; B01L 2300/0663; B01L 2300/0816; B01L 2300/0883; B01L 2300/0887

(57) ABSTRACT

Some embodiments disclosed herein relate to a hand-held electrochemical-sensor system integrated within a self-powered capillary microfluidic cartridge for quantitative and digital detection of target biomolecules and bioparticles, and devices and methods relating thereto. The system can allow for rapid detection of target biomolecules and bioparticles via one or more detection routes, simultaneously from biological samples such as tissues, bodily fluids, and/or the like. Target biomolecules and bioparticles include but are not limited to DNAs, RNAs, proteins, metabolites, exosomes, infectious agents, biproducts, nucleic acids, blood-born vectors, microbes (such as bacteria, viruses, fungi, protozoa, and/or the like), helminths, host immunoglobulins, and small molecules in different fluids or biofluids. Target bioparticles include cells, bacteria, pathogens, and viruses.

### RELATED APPLICATION

This patent application claims priority to U.S. provisional patent application No. 63/262,018 filed on Oct. 1, 2021, the entire disclosure of which is incorporated by reference.

### FIELD OF THE DISCLOSURE

The present disclosure relates generally to devices, systems, and methods for detecting target substances, and in particular to point-of-care devices and systems for detecting target biomolecules (proteins, metabolites, cytokines, nucleic acids, extracellular vesicles) and bioparticles (cells, bacteria, pathogens, viruses) in different bodily fluids (urine, blood, serum, plasma, sweat, tear, nasal, and saliva) and methods relating thereto.

### BACKGROUND

There has been an increasing demand for early-stage disease diagnosis, prognosis, and monitoring in the last decade. More importantly, the pandemic has increased the importance and need for diversified, point of care (POC), and individualized disease testing with the mandate of quantifying the disease state. Within POC applications, there is a pressing need in healthcare technologies for rapid and sensitive detection of target biomolecules and bioparticles such as DNAs, RNAs, cells, pathogens, viruses, bacteria, proteins, metabolites, exosomes, and small molecules in different biofluids, for diagnosing and digital monitoring of diseases and other health conditions. In industrial, environmental, and emergency/disaster relieve settings, there is also a need for on-site rapid and quantitative detection of target biomolecules and bioparticles, for example, for the purposes of air and water quality monitoring and chemical and biological threat assessment, to allow immediate remedial action and/or evacuation to take place.

Microbiological testing, for example, demonstrates how microfluidics may provide benefit in one respect wherein traditionally, the plating and culturing to determine cell counts of bacteria involves multiple biochemical and serological characterization steps typically involves days to weeks and hence is unsuitable for many applications. Accordingly, developments of alternatives including emerging technologies such as enzyme linked immunosorbent assay (ELISA), polymerase chain reaction (PCR), and flow cytometry have been geared to increasing speed of detection and reducing the volume of sample used. Pathogen detection utilizing ELISA has also become well established. However, integrated microfluidic systems (also referred to as “lab-on-a-chip”) offer improvements in the mass transport of the bacteria to the sensors and reductions in detection time to below 30 minutes.

A major challenge for the clinical use of “lab-on-a-chip” (LOAC) systems has been that they are generally complex and involve sophisticated peripheral equipment, and as a result have proven much more difficult than anticipated to implement as low cost, robust and portable systems. Whilst less integrated solutions have also been developed these are generally categorized as biosensors. Biosensors basically incorporate biological recognition elements (probes) such as antibodies, nucleic acids, aptamers, and other types of receptors to provide a specific affinity toward a target analyte, and a transducer that converts the ensuing recognition event and biochemical activity into a measurable signal (commonly optical or electrical in nature). A wide range of biosensors has been developed for selective biomolecule detection, but few can be practically integrated into LOAC systems.

Some electrochemical biosensors have screen-printed electrodes (SPEs) improved/modified with conductive nanomaterials and such SPE-based electrochemical biosensors have two to three orders of magnitude higher performance (detection range, limit of detection (LOD) and sensitivity) than ELISAs, a conventional alternative protein biomarker quantification technique. The process of electrochemical biosensor preparation consists of several steps, usually starting with modifying or improving the surface of electrode for e.g., with nanomaterial-based surface modification on the working electrode (WE), utilized to increase surface area, enhance detection performances, and to provide a suitable interaction with cross-linkers for detecting the molecules of target. The second step is to functionalize the modified WE with cross-linkers, adding amine or carboxyl groups suitable for immobilizing capture molecules (e.g., antibodies, aptamers, and peptides) and molecularly imprinted polymers (MIPs)). However, there are critical technical challenges related to preparation steps, which limits the translation of these biosensors from the lab into real clinical uses/application/implementation.

The complexity and multistep deposition of nanomaterials on SPEs, and scalable and reproducible creation of nanomaterials on electrodes are not consistent all the time, therefore, difficult to achieve electrode-level error-free response. Several electrode fabrication platforms have been developed to address this challenge, such as microfluidic chips producing microfibers and microcarriers, to create complex micro/nanomaterial constructs on the electrodes. Although these techniques are appropriate for laboratory-scale fabrication, their utility for mass-manufacturing is constrained by their high cost of fabrication, limited throughput performance, and need for several steps of electrodeposition and time-consuming rinsing and drying phases. The challenge to incorporate functionalization steps with e.g., the amine or carboxyl moieties further added over modified SPEs to selectively immobilize capture molecules (antibodies or aptamers). Surface functionalization methods add one or more manually-performed steps to the sensor preparation process, which adversely affect the reproducibility of biosensors, and involve hours of incubation.

Therefore, there exists a need for improved LOAC point-of-care biosensing technology for the detection of various target biomolecules and bioparticles that is portable, using compact possibly hand-held instruments; does not require expensive laboratory equipment, elaborate multi-step processes and reagents, and skilled personnel; and provides timely, and reliable results. There is also a need for biosensors that can be easily integrated into LOAC systems, are amenable to miniaturization and mass production, and are reusable or disposable. Moreover, the detection of biomarkers in complex bodily fluids mostly needs steps of sample preparation (cell or particle filtration, molecule extraction) that needs to be integrated into new LOAC systems.

### SUMMARY OF THE DISCLOSURE

According to an aspect, there is provided a sensor unit configured for use in an electrochemical-sensor system having a reader module and a microfluidic unit. The sensor unit has a distal portion configured to connect with the microfluidic unit for receiving a sample, a proximal portion configured to connect with the reader module for measuring electrochemical properties of the sample, and a substrate extending from the distal portion to the proximal portion and enabling fluid to flow thereon. The sensor unit also has a first working electrode distributed on the substrate and extending from the distal portion to the proximal portion, such that the first working electrode has a sampling end in the distal portion and a connecting end in the proximal portion, a second working electrode distributed on the substrate and extending from the distal portion to the proximal portion, such that the second working electrode has a sampling end in the distal portion and a connecting end in the proximal portion, and a control electrode distributed on the substrate and extending from the distal portion to the proximal portion, such that the control electrode has a connecting end in the proximal portion and wraps around the first working electrode and the second working electrode in the distal portion.

In accordance with an embodiment of the disclosure, the sampling end of the first working electrode and the sampling end of the second working electrode are separated from each other by a defined distance in a range of 125 to 750 μm. In addition, the first working electrode and the second working electrode have a combined surface area, and the control electrode has a defined surface area, such that a ratio of the combined surface area of the working electrodes to the defined surface area of the control electrode is in a range of 0.2 to 1.25. Notably, the combination of the defined distance and the ratio can help to mitigate or eliminate crosstalk between the working electrodes during operation.

According to another aspect, there is provided a combination including a microfluidic unit, and a sensor unit as summarized above. In some implementations, the sensor unit and the microfluidic unit are both part of a single integrated component which is a sensor module. In some implementations, the sensor module has an inlet reservoir in the form of a hole for receiving the sample, a side redox route wherein a presoaked redox reagents mixes with the sample, and a biosensing chamber wherein an antibody-antigen interaction occurs. In some implementations, the combination is an electrochemical-sensor system and further includes a reader module. By having the sensor unit as summarized above, the combination can likewise achieve the benefit of crosstalk mitigation.

According to another aspect, there is provided a sensor unit configured for use in an electrochemical-sensor system having a reader module and a microfluidic unit. The sensor unit has a distal portion configured to connect with the microfluidic unit for receiving a sample, a proximal portion configured to connect with the reader module for measuring electrochemical properties of the sample, and a substrate extending from the distal portion to the proximal portion and enabling fluid to flow thereon. The sensor unit also has a plurality of electrodes distributed on the substrate and extending from the distal portion to the proximal portion.

In accordance with an embodiment of the disclosure, each electrode includes a nanostructured-sensing surface having a plurality of capture areas, such that the nanostructured-sensing surface includes 10-20% w/w poly(3,4-ethylenedioxythiophene) polystyrene sulfonate (PEDOT:PSS)/Graphene nanocomposite and 80-90% w/w Graphite. Notably, the aforementioned proportions of the poly(3,4-ethylenedioxythiophene) polystyrene sulfonate (PEDOT:PSS) and Graphene nanocomposite can enable zero-step functionalization of the electrodes for reproducible immobilization of capture molecules and for sensitive and rapid detection of biomolecules and bioparticles.

According to another aspect, there is provided a combination including a microfluidic unit, and a sensor unit as summarized above. In some implementations, the sensor unit and the microfluidic unit are both part of a single integrated component which is a sensor module. In some implementations, the sensor module has an inlet reservoir in the form of a hole for receiving the sample, a side redox route wherein a presoaked redox reagents mixes with the sample, and a biosensing chamber wherein an antibody-antigen interaction occurs. In some implementations, the combination is an electrochemical-sensor system and further includes a reader module. By having the sensor unit as summarized above, the combination can likewise achieve the benefit of zero-step functionalization and reproducibility.

According to another aspect, there is provided a method that involves applying conductive ink onto a substrate such that, upon the conductive ink drying, an electrode having a nanostructured-sensing surface is formed on the substrate. In accordance with an embodiment of the disclosure, the conductive ink includes 0.2-0.3 mg/mL PEDOT:PSS mixed with 1-2 mg/mL electrochemically exfoliated graphene. According to another aspect, there is provided a conductive ink including 0.2-0.3 mg/ml PEDOT:PSS mixed with 1-2 mg/mL electrochemically exfoliated graphene. With the aforementioned concentrations, benefits of zero-step functionalization, reproducibility, and sensitive and rapid detection can be possible.

According to another aspect, there is provided a microfluidic unit. The microfluidic unit has a microchannel having a sample-receiving section configured to receive a sample, and an electrodes-interface section configured to supply the sample to electrodes of a sensor unit. In accordance with an embodiment of the disclosure, the microchannel has a gap between the sample-receiving section and the electrodes-interface section configured to slow down movement of the sample and thereby delay the supplying of the sample to the electrodes. Such delay can be useful, for example to enable more time for dissolving of presoaked molecules into the sample.

According to another aspect, there is provided a combination including a sensor unit, and a microfluidic unit as summarized above. In some implementations, the sensor unit and the microfluidic unit are both part of a single integrated component which is a sensor module. In some implementations, the sensor module has an inlet reservoir in the form of a hole for receiving the sample, a side redox route wherein a presoaked redox reagents mixes with the sample, and a biosensing chamber wherein an antibody-antigen interaction occurs. In some implementations, the combination is an electrochemical-sensor system and further includes a reader module. By having the microfluidic unit as summarized above, the combination can likewise achieve the benefit of more time for dissolving of presoaked molecules into the sample.

Other aspects and features of the present disclosure will become apparent, to those ordinarily skilled in the art, upon review of the following description of the various embodiments of the disclosure.

### BRIEF DESCRIPTION OF THE DRAWINGS

Embodiments will now be described with reference to the attached drawings in which:

FIG. 1A is a schematic perspective view of a portable electrochemical-sensor system, according to one embodiment of the present disclosure, the portable electrochemical-sensor system comprising a reader module, and a sensor module that comprises a sensor unit and a microfluidic unit.

FIG. 1B is a schematic plan view of the electrochemical-sensor system shown in FIG. 1A.

FIG. 2 is a schematic view of the sensor unit of the electrochemical-sensor system shown in FIG. 1A, where the sensor unit comprising a substrate and a plurality of electrodes, including a reference electrode (RE), a control electrode (CE), and two working electrodes (WE).

FIGS. 3A and 3B are schematic diagrams of the circuitries of the reader module shown in FIG. 1A for electrically coupling to the electrodes of the sensor unit shown in FIG. 2 for measuring one or more target biomarkers on the sensor unit.

FIG. 4 is a schematic view of the sensor unit shown in FIG. 2, illustrating the substrate and the WE, wherein the WE comprises a nanostructured-sensing surface embedded with poly(3,4-ethylenedioxythiophene) polystyrene sulfonate (PEDOT:PSS) and Graphene.

FIG. 5 is an exploded perspective view of the microfluidic unit shown in FIG. 1A.

FIG. 6 is a flowchart showing a process for using the electrochemical-sensor system shown in FIG. 1A to determine the presence of target biomarkers or detect their concentration.

FIG. 7 is a schematic view of an alternative sensor module usable in the electrochemical-sensor system of FIG. 1A, according to another embodiment of the present disclosure.

FIG. 8 is a schematic view of a bottom substrate layer of the sensor module of FIG. 7, where the bottom substrate layer comprising a substrate and an electrode, according to one embodiment.

FIG. 9 is a flowchart showing a process for screen printing of the electrode shown in FIG. 8 using intermixed Graphene@PEDOT:PSS-Carbon (GiPEC) conductive ink and the silver conductor for the lead part.

FIG. 10 is a schematic view of the 9-electrode design including 7 WEs, 1 Re, and 1 CE for measuring 7 bio-analyte simultaneously.

FIG. 11 is a graph comparing the reproducibility of the GiPEC SPE (GiPEC E) with the respective reproducibility of the pure carbon electrode (Carbon E) and DropSens SPEs (DRP-150 and DRP-110).

FIGS. 12A and 12B are schematics of a multiplex electrochemical-sensor system, according to one embodiment of the present disclosure, the electrochemical-sensor system comprising a reader module and sensor module which comprises a sensor unit.

FIG. 13 is a schematic plan view of the sensor unit of the electrochemical-sensor system shown in FIGS. 12A and 12B, where the sensor unit comprising a substrate and a plurality of electrodes in parallel including a RE, CE, and WE.

FIGS. 14A and 14B are graphs showing a typical applied DPV voltage and the resulting current from the DPV voltage.

FIGS. 15A and 15B are schematics of sample potentiostat circuitry comprising two op-amps configured to connect to the electrodes for one of the sensing strips.

FIG. 16 is a schematic view of one of the sensor unit of the electrochemical-sensor system shown in FIG. 13, where the sensor unit comprising a substrate and a plurality of electrodes including RE, CE, and WE.

FIG. 17 is a flowchart showing a process of fabricating nano-porous functionalized carbon electrode as the electrochemical-sensor system shown in FIG. 16 to determine the presence of target biomarkers or detect their concentration.

FIGS. 18A to 18D are graphs demonstrating selectivity versus specificity of the developed electrodes shown in FIG. 17 for diagnosis and prognosis of 107 clinical TBI, stroke and SCI samples compared to the SIMOA assay.

FIGS. 19A to 19C are graphs showing the capability of the developed sensor shown in FIG. 17 in measuring the change in GFAP concentrations over the course of weeks post-injury and as a quantitative indication of patient recovery.

FIG. 20 is a schematic plan view of an alternative sensor module usable in the electrochemical-sensor system of FIG. 7, according to another embodiment of the present disclosure.

FIG. 21 is a schematic plan view of the sequence of liquid flow in the platform assay shown in FIG. 20 automatically through which the self-powered microfluidic unit controls sequential delivery of the sample dispensed in the inlet well and the sensing reagents.

FIGS. 22A and 22B are graphs showing the filling optimization time of the sensing chamber and redox channel shown in FIG. 20 with the human serum and PBS.

FIG. 23 is a stepwise schematic view of the zinc oxide (ZnO)/reduced graphene oxide (rGO) modified electrode shown in FIG. 20 to measure the concentration of the target molecule.

FIGS. 24A to 24D show physical and chemical characterizations of the electrode's surface shown in FIG. 23.

FIG. 25 is a graph showing the electrochemical stability of the ZnO/rGO coated electrode shown in FIG. 23.

FIG. 26 is a graph showing the effect of the cross-linker used for immobilizing the bio-capture molecule on the ZnO/rGO shown in FIG. 23.

FIG. 27 is a graph showing the calibration curve of the ZnO/rGO coated electrochemical nano-biosensor shown in FIG. 23.

FIGS. 28A to 28C is a graph showing the selectivity and clinical testing of the developed electrode shown in FIG. 23.

FIG. 29 is an image of another self-powered microfluidics chip embedding the 2WE strip shown in FIG. 2 to determine the concentration of the biomolecule in a biofluid sample.

FIG. 30 is an image of another self-powered microfluidics chip embedding the 2WE strip shown in FIG. 2 to determine the concentration of the biomolecule in a biofluid sample.

FIG. 31 is an image of a portable electrochemical-sensor system, according to one embodiment of the present disclosure, the portable electrochemical-sensor system comprising a reader module and a sensor module which comprises a sensor unit and a microfluidic unit shown in FIG. 30.

FIG. 32 is an image of another self-powered microfluidic chip embedding the small/miniaturized 1WE strip for automated determination of the concentration of the biomolecule in a biofluid sample.

FIGS. 33A to 33C are schematic representations of a fabrication process of bio-ready sensing units containing simultaneous ultrasonic mixing and heating for graphene nanosheet delamination/exfoliation and infrared curing and cooling for sudden evaporation of solvents resulting in accumulation of the graphene nanosheets on the surface.

FIGS. 34A and 34B are schematics of simulations for sensor units having varying distances between working electrodes.

### DETAILED DESCRIPTION OF EMBODIMENTS

It should be understood at the outset that although illustrative implementations of one or more embodiments of the present disclosure are provided below, the disclosed systems and/or methods may be implemented using any number of techniques. The disclosure should in no way be limited to the illustrative implementations, drawings, and techniques illustrated below, including the exemplary designs and implementations illustrated and described herein, but may be modified within the scope of the appended claims along with their full scope of equivalents.

### Introduction

Some embodiments disclosed herein relate to a hand-held electrochemical-sensor system integrated within a self-powered capillary microfluidic cartridge for quantitative and digital detection of target biomolecules and bioparticles, and devices and methods relating thereto. The system can allow for rapid detection of target biomolecules and bioparticles via one or more detection routes, simultaneously from biological samples such as tissues, bodily fluids, and/or the like. Target biomolecules and bioparticles include but are not limited to DNAs, RNAs, proteins, metabolites, exosomes, infectious agents, biproducts, nucleic acids, blood-born vectors, microbes (such as bacteria, viruses, fungi, protozoa, and/or the like), helminths, host immunoglobulins, and small molecules in different fluids or biofluids. Target bioparticles include cells, bacteria, pathogens, and viruses.

In some embodiments, the system comprises a sensor module and a reader module. In some embodiments, the sensor module comprises an electrochemical-based sensing mechanism and a microfluidic mechanism. The microfluidic mechanism is configured for sample preparation and target extraction and purification while conducting the target and/or the like to the sensing mechanism for capturing and analyzing. The microfluidic mechanisms also automated the steps of electrochemical biosensing all within the same chip.

A collected sample may be applied through a sample port in the sensor module or on a designated surface of the sensor module. The applied sample may be filtered, lysed, purified, modified, and/or absorbed, and processed within the sensor module, as appropriate, to enhance, enrich, or modify the quality of its target detection. In some embodiments, the prepared sample may be further analyzed by the sensing mechanism of the sensor module. In some embodiments, the prepared sample may be analyzed via electrochemical-based methods to give a corresponding read when the sensor module is integrated with the reader module. In some embodiments, the prepared sample may be analyzed via electrochemical-based methods while an optical detection method is coupled to electrochemical sensing method to self-validate the sensor data.

In some embodiments, the reader module comprises an electronic reader which may comprise embedded components, a processor, a memory, data storage, and/or analog/digital circuitry. The electronic reader may also comprise inlet ports to communicate with the sensor module. The reader module may comprise a display and/or a user interface for user interfacing and may comprise communication mechanisms to transmit data to a computing device.

The biosensing technology disclosed herein may simultaneously detect multiple target biomarkers (biomolecules or bioparticles) (“multiplexing”) for either detection of multiple biomarkers of different types or to do self-validation of the sensing. In some embodiments, the system may comprise a combination of test kits, readout, software application programs or App, servers, and websites. The application programs may manage the data transfer from the reader module to a server for one or more of:
1) data collection,
2) communication with a Health Care Organization (HCO),
3) population screening,
4) remote feedback to patients on their health status, and
5) remote signals accessible to physicians to help them better decision-making remotely.

### Electrochemical-Sensor System

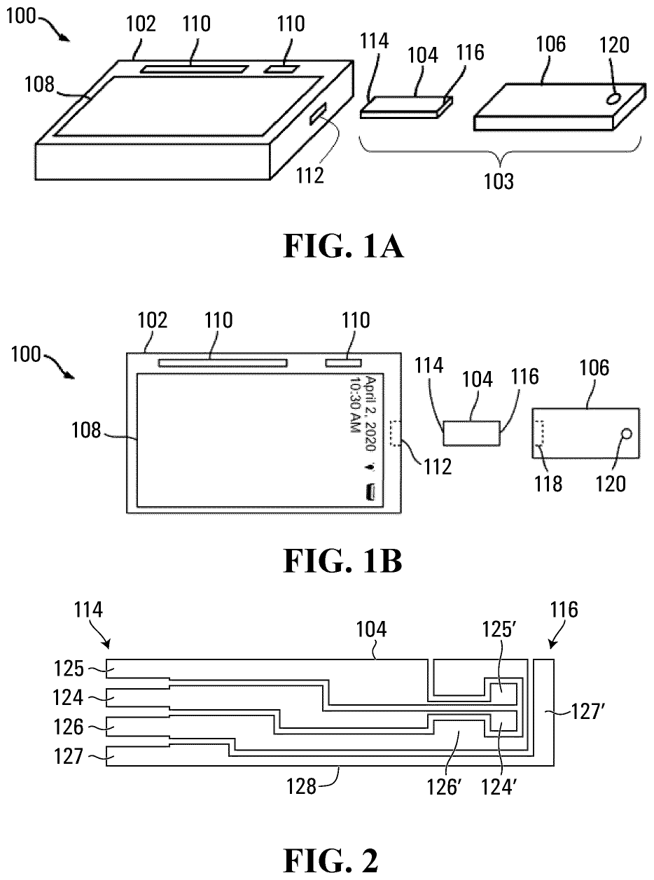

Turning now to FIGS. 1A and 1B, an electrochemical-sensor system is shown and is generally identified using the reference numeral 100, which may be used for analyzing, determining, and monitoring the presence of one or more target biomarkers (biomolecules and bioparticles) in a sample. In some embodiments, system 100 is configured to be portable and may be used for home-based testing for disease diagnosis and prognosis. Those skilled in the art will appreciate that the electrochemical-sensor system 100 may also be used in other places such as health centers, clinics, hospital, and the like, and for other applications such as industrial, environmental, emergency/disaster relieve, etc.

As shown in FIGS. 1A and 1B, the electrochemical-sensor system 100 in some embodiments comprises a reader module 102 in the form of a point-of-care (PoC) device which may be sized for portability for personal use; and a sensor module 103 comprising a sensor unit 104 which may be disposable and a microfluidic unit 106, for detecting a target antigen in a sample. For example, the detection of the target biomolecules/bioparticles may be used for detecting infectious agents and assessing a patient's health conditions with respect to a disease, wherein the presence, absence, or variation in the quantities of the target biomarker in the patient's bodily fluids may be used as an indicator or predictor of disease.

In some embodiments, the reader module 102 and the sensor unit 104 may be similar to those disclosed in Canadian Patent Application No. 3,060,849, entitled “PORTABLE ELECTROCHEMICAL-SENSOR SYSTEM FOR ANALYZING USER HEALTH CONDITIONS AND METHOD THEREOF”, filed on Nov. 4, 2019, the content of which is incorporated herein by reference in its entirety.

In the illustrated embodiment, the reader module 102 comprises a user interface, which may include an output mechanism such as a display or touch screen 108 and an input mechanism such as touch screen 108 and one or more buttons 110 for receiving user input. User input may include, for example, user instructions (e.g., turning the reader module 102 on or off, starting a diagnostic process, displaying readings obtained in the diagnostic process, displaying previous diagnostic readings, and/or the like) and/or user data (e.g., the user's age, sex, weight, height, and/or the like). Other configurations of the user interface are possible. The reader module 102 also comprises a receiving port 112 for receiving a proximal portion 114 of the sensor unit 104, a control structure (not shown) such as a RFduino microcontroller offered by RFduino Inc. of Hermosa Beach, CA, USA, and relevant circuitries. The reader module 102 may comprise a processor, memory, data storage, software, hardware, and/or firmware known to those skilled in the art. The reader module 102 also comprises a power source (not shown) such as a battery for powering various components thereof.

The circuitries of the reader module 102 may include an analysis circuitry such as a potentiostat circuitry for biosensing (described in more detail later) and a monitoring circuitry for other tasks such as performing user-instructed operations, detecting the insertion of the sensor unit 104, reading and displaying the measured levels of target antigens, storing measurement data, transmitting measurement data to a remote device for trend tracking, and/or the like.

As shown in FIG. 2, the sensor unit 104 may comprise a plurality of electrodes 124′ to 127′ distributed on a biocompatible and flexible substrate 128 that enables fluid to flow thereon. In particular, the sensor unit 104 comprises a reference electrode (RE) 127′, a control electrode (CE) 126′, and two working electrodes (WEs) 124′ and 125′ on the proximal portion 114 of thereof and extending into a lead region forming corresponding RE 127, CE 126 and WEs 124 and 125, for measuring the electrochemical properties of samples (not shown) received therein. In some implementations, the control electrode wraps around the first working electrode and the second working electrode in the distal portion.

In these embodiments, the surfaces of the WEs 124′ and 125′ may be modified or otherwise treated with a mediator to mediate the electron transfer from the electrodes to the sample fluids. Different WEs 124′ and 125′ may be configured to harbor different elements for detecting one or more different target biomarkers. The sensor unit 104 also comprises a pair of identification electrodes 126 and 127 on the proximal portion 114 thereof, which can become joined by a trace with a predefined resistance or a predefined impedance of the reader module 102 upon insertion, for indicating the type of target biomarkers that the sensor unit 104 is configured to detect.

As described above, the analysis circuitry of the portable reader module 102 may be designed to be corresponding to the circuitry of the sensor unit 104 for monitoring the electrochemical reaction between the target biomarker(s) in the samples and the electrodes 124′ to 127′ on the sensor unit 104. With the sensor unit 104 shown in FIG. 2, the analysis circuitry of the portable reader module 102 correspondingly comprises a set of coupling electrodes in the receiving port 112 for electrically engaging the electrodes 124 to 127 of the sensor unit 104.

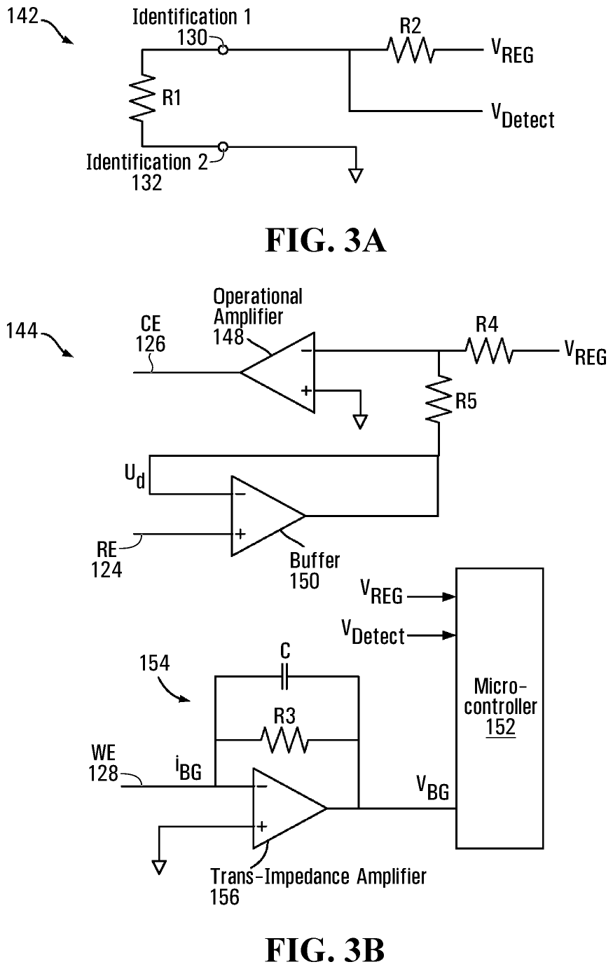

With reference to FIGS. 3A and 3B, the reader module 102 comprises a plurality of circuits 142 and 144 for electrically engaging the electrodes 124 to 127 when the proximal portion 114 of the sensor unit 104 is inserted into the receiving port 112.

As shown in FIG. 3A, a first circuit 142 in the form of a voltage divider is used for determining the type of target antigen. As described above, in some embodiments, the identification electrodes 130 and 132 (corresponding to the identification electrodes 126 and 127 of the sensor unit 104) have a predefined resistance there between which is represented by a resistor R₁. The resistance of R₁ is predefined and indicative of the type of target biomarker that the sensor unit 104 is suitable to detect. The first circuit 142 electrically engages the identification electrodes 130 and 132 and applies a voltage V_{REG} (e.g., 3.3V) thereto via a resistor R₂ with known resistance. A voltage signal V_{Detect} is outputted from between R₁ and R₂. Therefore, V_{Detect}=V_{REG}R₁/(R₁+R₂), and the resistance of R₁ and in turn the type of target antigen may be detected by comparing V_{Detect} with V_{REG}.

As shown in FIG. 3B, a second circuit 144 in the form of a Direct-Current (DC) potentiostat circuit is used to control the voltage between the WE 128 and RE 124. In the illustrated embodiments, the samples on the sensor unit 104 act as the electrolyte between the WE 128 (acting as a cathode), the RE 124 (acting as an anode), and the CE 126. Because of the nature of the operational amplifier (op-amp) 148, a current is supplied through the CE 126 until the voltage at RE 124 and U_{d} is the same. Thus, U_{d} determines the voltage of the electrolyte, and consequently determines the accuracy of target biomarker measurements as a too-low U_{d} may not be able to generate a sufficient measurement resolution and a too-high U_{d} may trigger inferencing reactions or surface property changes. Thus, in the second circuit 144, the three-electrode configuration is connected to a DC potentiostat circuitry wherein a constant DC voltage is regulated and applied over the WE 128 and RE 124 of the sensor unit 104. The circuit 144 may be used for determining the electrochemical properties of a sample fluid for analysis of the sample fluid by detecting and determining impedimetric measurements.

As shown in FIG. 3B, a control structure 152 (e.g., a microcontroller) compares V_{Detect} with V_{REG} and determines the type of target biomarker. The microcontroller 152 then adjusts the biosensing parameters (such as U_{d}) to adapt to the determined type of target antigen and measures the voltage of WE 128. An amplifying circuit 154 which in the illustrated embodiment comprises an amplifier 156, a resistor R₃, and a capacitor C is used to amplify the signal of WE 128. In this way, the electrochemical properties of the target biomarker in the samples on the sensor unit 104 are measured and may be used, for example, to determine the patient's health conditions.

In some embodiments, the DC potentiostat circuit may be coupled to a frequency-response analyzer used as an impedance-analysis system. For example, the system may contain four stages: current-to-voltage conversion with a multiplexer, an amplifier to accommodate extra electrodes within the system, a gain stage, and an eventual frequency-response analyzer implemented as an integrated circuit (IC). In various embodiments, the multiplexer may be used to switch the system between a calibration mode and one or more multiple-electrode modes.

Those skilled in the art will appreciate that the circuit 144 may also be used for the amperometric type of measurements which is commonly used in glucose detection. Moreover, the potentiometric type of measurements may also be implemented using the three-electrode configuration, wherein an Alternate-Current (AC) wave with a predefined frequency is applied for stimulating the samples while forward (e.g. by increasing the voltage) and reverse (e.g. by decreasing the voltage) current is measured to yield a differential current (forward-reverse).

Referring back to FIG. 2, in accordance with an embodiment of the disclosure, the sampling end 124′ of the first working electrode 124 and the sampling end 125′ of the second working electrode 125 are separated from each other by a defined distance in a range of 125 to 750 μm. In specific implementations, the defined distance is 300 μm, plus or minus some margin such as 10 μm for example. With increasing the defined distance between the two WEs (750 μm), a gradual potential difference is generated in the area between the two WEs, resulting in an unnecessary current flow in this area and a risk of noise generation. For the design with 125 μm distance between the WEs, it was observed that the upper and lower edges of the WEs allow for a higher current flow to the CE compared to the side edges, which is not desirable given that it generates inconsistencies in the electrical current of the side edges and the top edge. To ascertain that all areas of the WEs are involved equally in the sensitive signal generation, it is desirable to have a uniform current flow throughout all edges, as observed in the design with 300 μm distance between the WEs.

In addition, the first working electrode 124 and the second working electrode 125 have a combined surface area, and the control electrode 126 has a defined surface area, such that a ratio of the combined surface area of the working electrodes 124 and 125 to the defined surface area of the control electrode 126 is in a range of 0.2 to 1.25. In specific implementations, the ratio is 0.25 which is what is depicted in FIG. 2. Note that the ratio can be increased up to about five times what is depicted. Conversely, the ratio can be decreased, for example about 20%, from what is depicted.

Notably, the combination of the defined distance and the ratio can help to mitigate or eliminate crosstalk between the working electrodes 124 and 125 during operation. Any suitable combination of the defined distance and the ratio can be implemented based on the ranges mentioned above to achieve the benefit of crosstalk mitigation.

In some implementations, the first working electrode 124 and the second working electrode 125 have identical surface area. In other words, a defined surface area of each working electrode is half of the combined surface area of the working electrodes. However, other implementations are possible in which the defined surface area of the first working electrode 124 is different from the defined surface area of the second working electrode 125.

In some implementations, the first working electrode 124 and the second working electrode 125 are separated from each other by the defined distance between the proximal portion 114 and the distal portion 116. In specific implementations, the defined distance between the first working electrode 124 and the second working electrode 125 is constant between the proximal portion 114 and the distal portion 116. However, other implementations are possible in which the distance between the first working electrode 124 and the second working electrode 125 can vary between the proximal portion 114 and the distal portion 116.

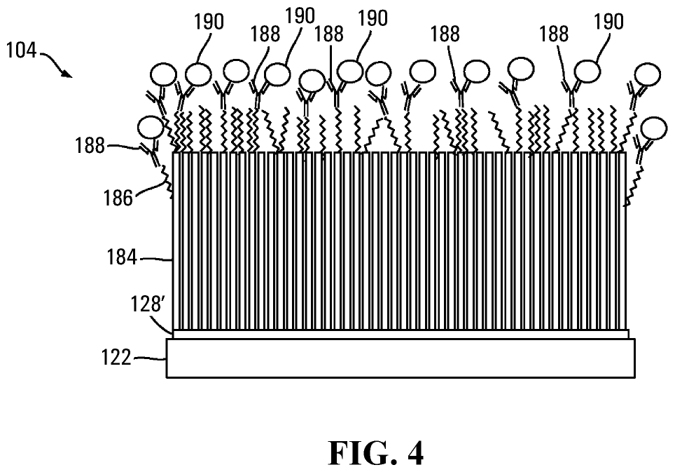

FIG. 4 is a schematic view of the sensor unit 104 showing the substrate 122 and the electrode WE 124′. As shown, the sensor unit 104 comprises a nanostructured-sensing surface in the sampling region 134 thereof for amplifying the amount of target antigen binding to the sensor unit 104 in order to achieve improved sensitivity. In one embodiment, the distal-side electrode WE 124′ comprises a nanostructured-sensing surface 182 having a plurality of capture areas 184 with poly(3,4-ethylenedioxythiophene) polystyrene sulfonate (PEDOT:PSS) and Graphene nanocomposite.

In some embodiments, the capture areas 184 are coated with molecules 188 of a specific type of detection element such as one or more immobilized capture ligand such as antibodies, enzymes, nucleic acid aptamers, and the like, for detecting a target biomarker 190 for which the detection element has a high specificity and affinity. The capture areas 184 are also coated with crosslinking molecules 186 which immobilize the detection element molecules 188 on to the capture areas 184 for capturing and reacting with the corresponding target biomarker 190.

When samples are received into a sampling region of the distal portion 116 (FIG. 2) of the sensor unit 104, electrochemical interaction between the target biomarker in the samples and the detection element molecules 188 occur and cause energy changes. When the sensor unit 104 is engaged with the ex-vivo reader module 102, the reader module 102 imparts energy to the sample fluid and measures the electrochemical properties thereof to generate a sample-fluid reading indicative of the concentration of the target biomarker in the sample fluid. The imparted energy may be electrical energy and the measured energy property may be the potential difference, current, or impedance.

Referring again to FIGS. 1A and 1B, the microfluidic unit 106 comprises a strip-receiving port 118 for receiving the distal portion 116 of the sensor unit 104 and an applicator-receiving port 120 for receiving a sampling head of an applicator such as a swab, pipettor, stool collector unit. In the illustrated embodiments, the strip-receiving port 118 and the applicator-receiving port 120 are arranged on opposite sides of the microfluidic device 106 along a longitudinal axis thereof.

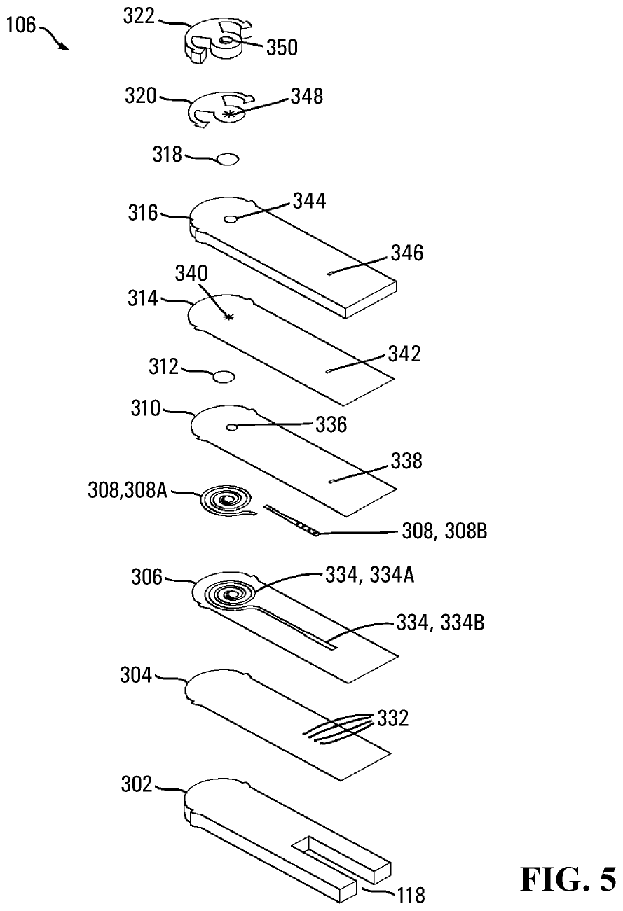

FIG. 5 is an exploded view of the microfluidic unit 106. As shown, the microfluidic unit 106 comprises a plurality of layers 302 to 322 made of suitable materials. For ease of illustration, the layers 302 to 322 are shown in FIG. 5 in a two-dimensional (2D) form. However, those skilled in the art will appreciate that each of the layers 302 to 322 may have a respectively height or thickness suitable for assembling to the microfluidic unit 106.

As shown, the microfluidic unit 106 comprises a bottom layer 302 made of a suitable material such as plastic, acrylic, or polyurethane. The bottom layer 302 has defined therein an opening or slot forming the strip-receiving port 118 for receiving the distal portion 116 of the sensor unit 104.

On top of the bottom layer 302, the microfluidic device 106 comprises a mask 304 having a plurality of electrode-holes 332. A plurality of electrodes (not shown) sealably extend through the electrode-holes 332 into an electrodes-interface section 334B of a microchannel 334 in the microchannel layer 306 (described in more detail later) for electrically coupling the electrodes 124′ to 127′ of the sensor unit 104 to the sample solution in the microchannel 334. Herein, the samples solution is obtained from samples such as body cells, tissue, bodily fluid, and/or the like obtained from a patient using a suitable applicator such as a swab, stool collector, or biofluid pipettor/fitter (described in more detail later).

An adhesive microchannel layer 306 is positioned on top of the mask 304. The microchannel layer 306 comprises a microchannel 334 for receiving therein a hydrophilic layer 308 and for creating a specific area around the electrodes 124′ to 128′ extended thereinto. In some embodiments, the microchannel 334 is manufactured using laser cut or injection molding.

As shown, the microchannel 334 comprises a spiral sample-receiving section 334A extending to an electrodes-interface section 334B. The spiral sample-receiving section 334A is positioned under the applicator-receiving port 120 for receiving the samples from a sampling applicator inserted into the applicator-receiving port 120. The electrodes-interface section 334B is positioned above the electrode-holes 332 and open thereto for receiving the tips of the electrodes 124′ to 127′.

In some embodiments, the hydrophilic layer 308 is made of a suitable coating hydrophilic fiber such as nitrocellulose and is presoaked with cell disruption buffer or ionic reagents to enable cell lysing, nucleic acids transport, and protein and cell content capture there along. The hydrophilic fiber has a protein capturing property and is tuned for its electric charge. The hydrophilic layer 308 comprises a spiral sample-receiving section 308A with a lysis buffer, and an electrodes-interface section 308B presoaked in redox probe for electrochemical sensing.

The spiral sample-receiving section 308A and the electrodes-interface section 308B have suitable and dimensions corresponding to those of the microchannel sections 334A and 334B for positioning thereinto. Moreover, the spiral sample-receiving section 308A and the electrodes-interface section 308B are positioned in the microchannel 334 with a suitable gap (e.g., several millimeters, 2 to 5 mm) therebetween. This gap provides delays needed for dissolving of presoaked molecules into the flowing samples and can be easily tuned by proper positioning of the fiber within the channels.

For ease of illustration, the electrodes-interface section 308B shown in FIG. 5 marks the electrode-contact positions. However, those skilled in the art will appreciate that in practice, the electrodes-interface section 308B does not need to have such marks.

After the hydrophilic layer 308 is positioned into the microchannel 334, a protective layer 310 is coupled to the top of the microchannel layer 306 for protecting the microchannel 334 and hydrophilic layer 308. The protective layer 310 comprises a sampling hole 336 positioned above the spiral sample-receiving section 334A of the microchannel 334 for transporting the released target biomarker on to the spiral sample-receiving section 308A of the hydrophilic layer 308, and a vent hole 338 positioned above the electrodes-interface section 334B thereof.

A lower barrier layer 312 in the form of a burst disk is then coupled to the top of the protective layer 310 at the position above the sampling hole 336 of the protective layer 310. Then, an adhesive protective layer 314 is coupled to the protective layer 310 sandwiching the lower barrier layer 312 therebetween. The adhesive protective layer 314 comprises a valve 340 at a position corresponding to the sampling hole 336 of the protective layer 310, and a vent hole 342 at a position corresponding to the vent hole 338 of the protective layer 310. The valve 340 comprises one or more flexible pieces forming one or more slits which are openable under a pressure applied by a swab/pipettor inserted thereto, and closable when the swab/pipettor is removed therefrom.

A reservoir layer 316 is coupled to the top of the adhesive protective layer 314. The reservoir layer 316 comprises a reservoir chamber 344 in the form of a hole at a position corresponding to the valve 340 of the adhesive protective layer 314 and a vent hole 346 at a position corresponding to the vent hole 342 of the adhesive protective layer 314. The reservoir chamber 344 contains a harvest buffer (e.g., a preserving buffer and beads) for preventing the growth of bacteria on the applicator and maintaining the salinity needed for cells.

An upper barrier layer 318 in the form of a burst disk is positioned above the reservoir chamber 344 of the reservoir layer 316 for keeping the reservoir chamber 344 sterilization intact. An attachment layer 320 is then coupled to the reservoir layer 316 sandwiching the upper barrier layer 318 therebetween. The attachment layer 320 comprises a valve 348 at a position corresponding to the reservoir chamber 344 of the reservoir layer 316. Similar to the valve 340, the valve 348 comprises one or more flexible pieces forming one or more slits which are openable under a pressure applied by a swab/pipettor inserted thereto, and closable when the swab/pipettor is removed therefrom.

A topmost protective layer 322 is then coupled to the attachment layer 320. The topmost protective layer 322 comprises a guide 350 in the form of a hole at a position corresponding to the valve 348 of the attachment layer 320 for receiving a sampling swab and guiding the sampling swab therethrough.

In some embodiments, the layers 302 to 322 are coupled to one another by adhesives. In some embodiments, the layers 302 to 322 are made of flexible materials so that the microfluidic unit 106 may be incorporated into wearable biosensing systems. In some embodiments, one or more of the layers 302 to 322 may be made of acrylic and/or plastic.

While the sensor unit 104 and microfluidic unit 106 of the sensor module are shown as separate components in the illustrated embodiment, the sensor unit 104 and the microfluidic unit 106 may be assembled into a single integrated component or in a modular format in other embodiments. In some implementations, as a single integrated component, the sensor module is an LOAC system.

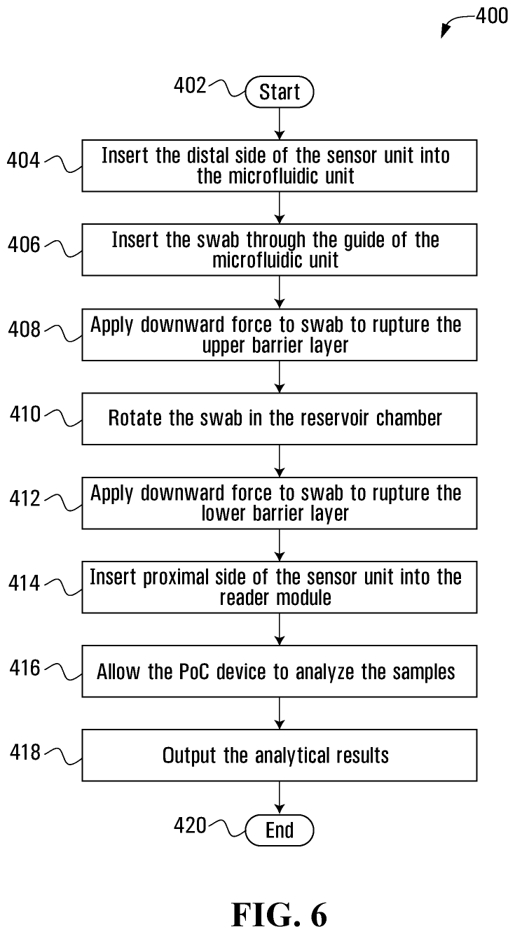

FIG. 6 is a flowchart showing a process 400 for using the portable electrochemical-sensor system 100 to determine for example infection agent of an infectious disease.

The process 400 starts (step 402) after an operator uses a sampling swab (not shown) and collects the samples such as nasal samples of a patient into the sampling head of the swab.

At step 404, the operator inserts the distal portion 116 of a sensor unit 104 into the strip-receiving port 118 of the microfluidic unit 106. Then, the operator inserts the sampling head of the swab through the guide 350 of the topmost protective layer 322 against the valve 348 of the attachment layer 320 (step 406).

At step 408, the operator applies a downward force to rupture the upper barrier layer 318 and forces the valve 348 of the attachment layer 320 to open so as to position the sampling head of the swab into the reservoir chamber 344 of the reservoir layer 316.

At step 410, the operator rotates the swab in the reservoir chamber 344 of the reservoir layer 316 for a predefined period of time such as about two (2) to three (3) minutes to ensure mixing with the harvest buffer to release target antigens from the applicator.

After rotating the swab, the operator applies another downward force (e.g., a downward force greater than that ruptured the upper barrier layer 318) to rupture the lower barrier layer 312 and forces the valve 340 of the adhesive protective layer 314 to open (step 412) so as to allow the samples solution containing the target antigens to move through the opened valve 340 of the adhesive protective layer 314 and the sampling hole 336 of the of the protective layer 310 into the spiral sample-receiving section 308A of the hydrophilic layer 308 in the microchannel 334 of the microchannel layer 306.

The operator then inserts the proximal portion 114 of the sensor unit 104 into the receiving port 112 of the reader module 102 (step 414) to allow the reader module 102 to analyze the samples.

With the capillary properties thereof, the hydrophilic layer 308 guides the samples solution to flow in the microchannel 334 from the spiral sample-receiving section 334A towards the electrodes-interface section 334B while going through the process of cell lysis and capture of specific biomolecules/nucleic acids. The aforementioned airgap between the spiral sample-receiving section 308A and electrodes-interface section 308B of the hydrophilic layer 308 delays the delivery of collected solution from the spiral sample-receiving section 308A to the and electrodes-interface section 308B and provides sufficient time for the interaction of the sample's solution and fiber molecules of the hydrophilic layer 308.

The hydrophobic and hydrophilic properties of the materials (e.g., Polyethylene terephthalate) of the hydrophilic layer 308 can be selected to control the flow pattern and/or flow rate of the samples solution along the microchannel 334, thereby predetermining the time it takes for the samples solution to flow from the spiral sample-receiving section 308A to the electrodes-interface section 308B.

After a suitable period (e.g., about 5 minutes), the electrodes-interface section 308B of the hydrophilic layer 308 receives the samples solution from the spiral sample-receiving section 308A and quickly (e.g., in a few seconds) delivers the samples solution to the electrodes (e.g., RE 127′, CE 126′, and WEs 124′ and 125′) in contact therewith.

The samples solution delivered to the electrodes consists mainly of nucleic acids, minimal proteins and cell content, and redox probe. The WEs 124′ and 125′ are already functionalized with capture probes for rapid detection of the target antigen, for example genetic material, proteins, biomolecules, and/or metabolites of an infectious agent. After a sufficient period of time (e.g., about five (5) to 20 minutes) of the samples solution interaction with the electrodes 124′ to 128′, the reader module 102 records the electrochemical signals of the electrodes 124 to 128 and detects the concentration of the target antigen.

After analyzing the samples, the analytical results may be output to a suitable target such as the display 108 of the reader module 102, a client-computing device (not shown) and/or a server computer (not shown) functionally connected to the reader module 102, and/or the like (step 418) and the process 400 ends (step 420). Herein, the connection between the reader module 102 and the client-computing device and/or server computer may be established using suitable wired and/or wireless communication technologies such as Ethernet, WI-FI®, (WI-FI is a registered trademark of Wi-Fi Alliance, Austin, TX, USA), BLUETOOTH® (BLUETOOTH is a registered trademark of Bluetooth Sig Inc., Kirkland, WA, USA), ZIGBEE® (ZIGBEE is a registered trademark of ZigBee Alliance Corp., San Ramon, CA, USA), 3G, 4G and/or 5G wireless mobile telecommunications technologies, parallel ports, serial ports, USB connections, optical connections, and/or the like.

The entire process 400 of sample collection from swab to the output of results can be performed using the system 100 without any manual sample preparation, centrifuge, pipetting, aliquoting, filtration, precipitation, and elution steps. In some embodiments, using the portable electrochemical-sensor system 100 to detect target biomarkers via process 400 can be performed in about 20 minutes.

The above-described portable electrochemical-sensor system 100 is suitable for detecting DNA or RNA viral using a PCR-equivalent process. In some embodiments, the portable electrochemical-sensor system 100 may be formed as an DNA or RNA-diagnostic kit for detecting infectious diseases such as viruses and bacteria.

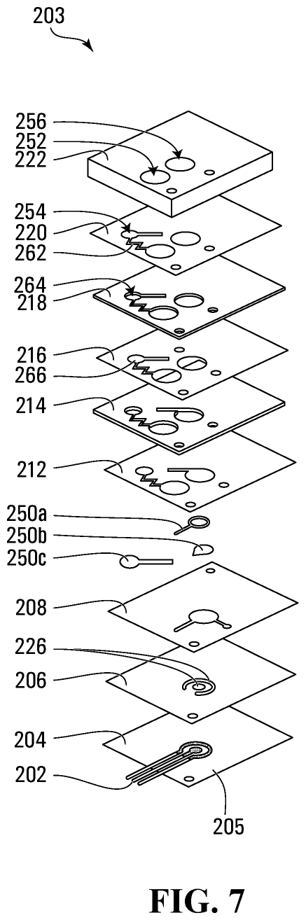

FIG. 7 is an exploded view of a sensor module 203 that can be used in the electrochemical-sensor system 100, in place of sensor module 103. As shown, the sensor module 203 comprises a plurality of layers 204 to 222 made of suitable materials. For ease of illustration, some of the layers 202 to 220 are shown in FIG. 7 in a two-dimensional (2D) form. However, those skilled in the art will appreciate that each of the layers 204 to 222 may have a respectively suitable height or thickness.

As shown, the sensor module 203 has a bottom substrate layer 204 comprising a substrate 205 and an SPE 202 printed on the substrate 205. In some implementations, the bottom substrate layer 204 is a PET sheet substrate. The bottom substrate layer 204 may be referred to as the sensor unit of the sensor module 203. In some embodiments, the sensor module 203 may comprise a lamination sheet (not shown) under the substrate 205. Layers 206 to 222 may be collectively referred to herein as the microfluidic unit of the sensor module 203. The microfluidic unit is configured to automate the electrochemical sensing steps. Each layer is described in detail below.

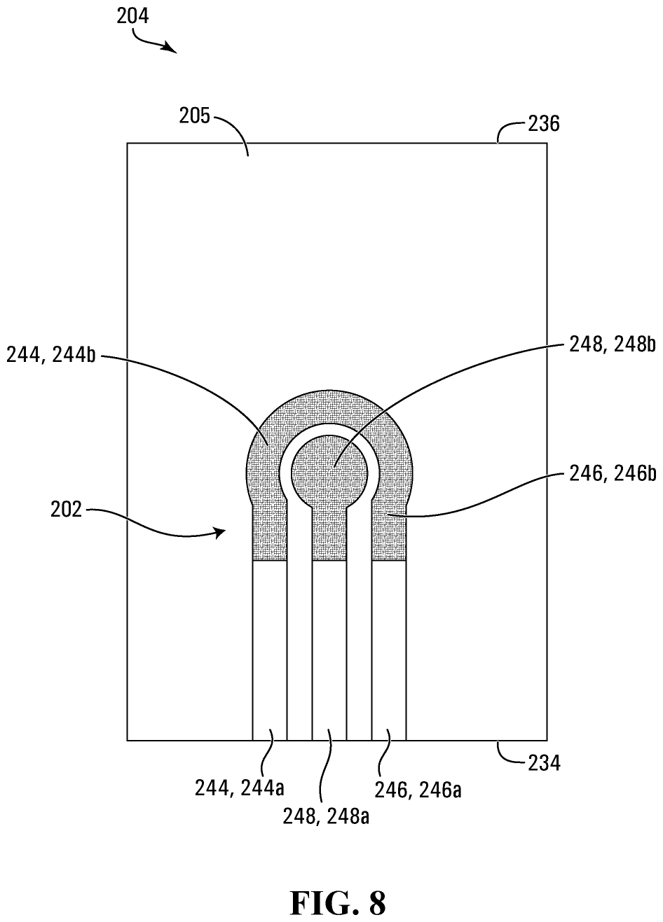

FIG. 8 shows the substrate 205 and the SPE 202 of the sensor unit, according to some embodiments. The SPE 202 aims to provide an increased surface area and conductivity compared to prior art SPEs and an enriched functionalized moieties for ultrasensitive detection of antigens, such as Glial Fibrillary Acidic Protein (GFAP), a known diagnostic biomarker of Central Nervous System (CNS) injuries; Nucleocapsid Protein and spiked proteins of SARS-CoV-2 as known antigens of COVID-19 diagnostics, or CD9, CD81, CD63 and TGF-beta expressing exosomal nanoparticles as known biomarkers of cancer exosomes, without the need for any intermediate step of nanomaterial coating or functionalization.

The substrate 205 may be made of a suitable material such as poly(ethylene terephthalate) (PET) or glass. In some embodiments, the substrate 205 is made of a flexible material for ease of integration with a microfluidic unit (described in detail below) of the sensor module 203. In some embodiments, the SPE 202 is fabricated using a novel ink derived from a mixture of Graphene Intermixed poly(3,4-ethylenedioxythiophene) Polystyrene sulfonate (PEDOT:PSS) ink and carbon ink. The ink mixture may be referred to as intermixed ink. In some embodiments, the surface chemistry of the SPE 202 contains the carboxyl or amine functional groups needed for capturing antibodies corresponding to a target antigen on a highly conductive electrode. In some embodiments, crosslinkers like L-Cysteine can be added on the printed ink to increase the density of carboxyl functional groups needed for further immobilization of antibody capture molecules.

In the illustrated embodiment, the substrate layer 204 has a proximal side 234 and distal side 236. The SPE 202 has three electrodes: a reference electrode (RE) 244, a control electrode (CE) 246, and a working electrode (WE) 248. The RE 244, CE 246, and WE 248 each has a respective conductor lead portion 244a,246a,248a, and a respective sensor portion 244b,246b,248b. In the illustrated embodiment, the sensor portions 244b,246b,248b are located further away from the proximal side 234 of the substrate 205 than the conductor lead portions 244a,246a,248a. In some embodiments, the conductor lead portions 244a,246a,248a are printed with a silver or created from conductive copper ink by temperature-controlled curing and selective laser writing techniques.

In some embodiments, the sensor portions 244b,246b,248b are printed with intermixed ink comprising about 10-20% w/w Graphene@PEDOT:PSS ink and about 80-90% w/w base carbon ink that are uniformly mixed together. In some embodiments, the Graphene@PEDOT:PSS ink consists of 0.2-0.3 mg mL⁻¹ PEDOT:PSS mixed with 1-2 mg mL⁻¹ electrochemically exfoliated graphene. As a skilled person can appreciate, intermixed ink with different proportions of Graphene@PEDOT:PSS ink and carbon ink may be used to fabricate the SPE 202 in other embodiments. However, it was found that increasing the weight percentage of the Graphene@PEDOT:PSS ink decreased the conductivity of the SPE and the consistency of the SPE due to the reduced viscosity and printability of the resulting intermixed ink.

Compared to a pure base carbon ink SPE (“carbon SPE”), the SPE 202 printed with intermixed ink (“GiPEC SPE”) showed more asperities of fragments and thus a higher surface roughness. The GiPEC SPE also has a higher hydrophilicity than the carbon SPE. Still further, it was found that the surface of the GiPEC SPE has carboxyl or amine functional groups, which are substantial elements that lead to the formation of covalent amidic binding between the SPE electrodes and the aminated bio-capture molecules. The presence of carboxyl functional groups on the GiPEC SPE eliminates the need for additional cross-linkers usually needed for immunosensor development. The applicant also found that the GiPEC SPE performed better in antibody (e.g. GFAP antibody) immobilization compared to (3-Aminopropyl)triethoxysilane N-Hydroxysuccinimide-1-ethyl-3-carbodiimide hydrochloride functionalized GiPEC SPEs. The GiPEC SPE therefore has a high surface area and active functional groups for high-caliber antibody immobilization compared to prior art SPEs. The material and process for making the GiPEC SPE inherently adds carboxyl functional groups to the GiPEC SPE, thereby eliminating the need for further cross-linking steps and accordingly reducing the fabrication time. In some embodiments, the GiPEC SPE described herein does not require any additional functionalization step and can be converted into a highly reproducible and sensitive biosensor by a single step drop casting of an antibody on its electrode surface.

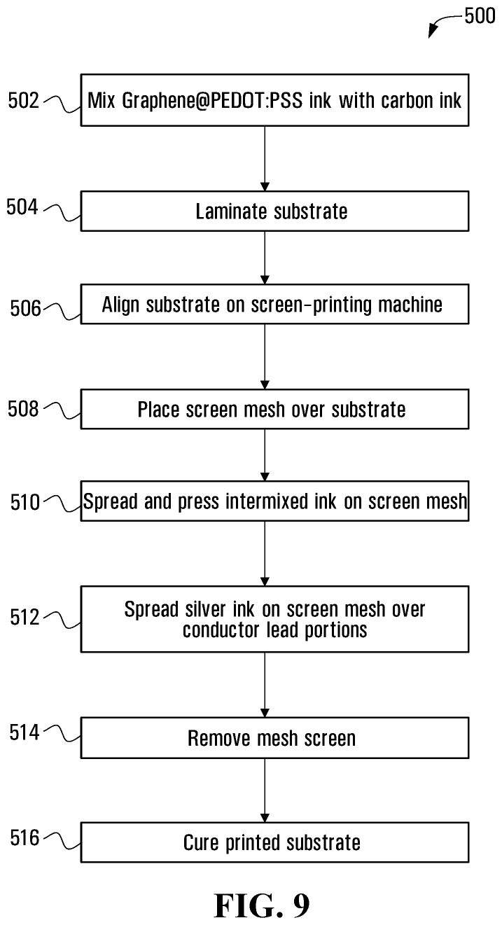

FIG. 9 illustrates a sample process 500 for making the SPE 202 according to some embodiments. The process 500 starts at step 502 wherein Graphene@PEDOT:PSS hybrid ink dispersed in Dimethylformamide (DMF) (Sigma, USA) is mixed with carbon ink (7102, Dupont, USA) to provide an intermixed ink. In some embodiments, the composition of the intermixed ink is about 10% w/w Graphene@PEDOT:PSS hybrid ink dispersed in DMF and about 90% w/w carbon ink. In some embodiments, the inks are mixed by mechanical stirring with a rotational speed of about 100 rpm for about 1 hour. While various w/w ratios of the Graphene@PEDOT:PSS in carbon ink were examined within 5%-30% w/w, the signals collected for 5-20% w/w mixture of Graphene@PEDOT:PSS in the base carbon ink showed reliable results, but the ink with the ratio percentage of higher than 20% showed to be non-screen-printable.

At step 504, a substrate sheet is laminated to provide substrate 205. In some embodiments, the substrate sheet comprises two layers of PET substrate (e.g. ST505, Melinex_{R}) attached to one another by an adhesive (e.g. 467MP adhesive transfer tape, 3M, USA). In some embodiments, the substrate 205 has a thickness of about 0.012″.

At step 506, the substrate may have channel features or prefabricated electronic layers, and therefore is aligned on a screen-printing machine, which may be, for example, the Micro Flatbed Printer (A.W.T. World Trade, Inc., USA).

At step 508, a screen mesh is placed over the substrate. The screen mesh has at least one cut-out in the shape of the electrodes 244,246,248 of the SPE 202. In some embodiments, the screen mesh is in the form of a polyester mesh with about 295 mesh/inch and about 48 μm thread diameter.

At step 510, the intermixed ink is spread on the screen mesh to print all three electrodes 244,246,248 of the SPE 202. The intermixed ink is pressed during the screen printing.

At step 512, silver ink is spread on the screen mesh to cover the conductor lead portions 244a,246a,248a of the SPE 202.

At step 514, the mesh screen is removed when the printing is done, leaving a printed substrate with wet electrodes.

At step 516, the printed substrate is cured with infrared light to provide one or more finished SPEs 202, depending on the number of cut-outs in the screen mesh. In some embodiments, the printed substrate is cured in a temperature-controlled process (steady temperature of about 100-120° C.) on an InfraRed curing machine (e.g. Customized Natgraph Air Force Dryer, Natgraph Ltd, UK) with the roller speed of about 10 ft min⁻¹, temperature units of about 100° C., 110° C., 120° C., and 140° C. for overall duration of about 10-15 min. The temperature-controlled curing process enables a gradual evaporation of the ink-binding solvents and prevents formation of amorphous rough surfaces and micro-cracks.

Process 500 thus allows multiple SPEs or SPEs with multiple working electrodes to be fabricated simultaneously.

In some embodiments, to help ensure a consistent formation of the printed ink on the substrate in terms of the thickness of the SPE's conductive material, a Digimatic indicator (e.g., Mitutoyo ID-H0530E, Mitutoyo America Corporation, USA) may be used to perform quality control by measuring the thickness of the printed ink in various spots on the SPE. In some embodiments, a finished SPE 202 printed with an intermixed carbon and 10% Graphene@PEDOT:PSS ink (“GiPEC 10% ink”) has an average thickness of 7±1 μm; and a finished SPE 202 printed with an intermixed carbon and 20% Graphene@PEDOT:PSS ink (“GiPEC 20% ink”) has an average thickness of 7±2 μm.

In some embodiments, to customize the interaction of the electrode with specific type of biomolecule analytes, the Graphene@PEDOT:PSS ink may be replaced with an optimized mixture of Quantum dots (CdS, ZnS, PbS, Mix of them, graphene); Graphene-based nanosheets (graphene nanosheet, reduced graphene oxide, graphene oxide); Carbon nanotubes (Single and Multi-Walled carbon nanotubes (SWCNT and MWCNT)); Metal oxide (Tungsten oxide, Aluminum-doped zinc oxide, Zinc oxide, Ruthenium Oxide, Nickle Oxide, Molybdenum oxide, Bismuth Oxide, Titania); Metal nanoparticles (Platinum, Palladium, Silver, Gold, Copper); Conductive polymers (Polyaniline, PEDOT:PSS, PEDOT:PSS@graphene, Polypyrole); or Carbon nanofibers. The solvent, density, viscosity, conductivity, thickness, and mechanical properties of these inks are tuned to customize them for detecting the target molecule/particle analyte within desired sensitivity, dynamic detection range, and limit of detection.

Table 1 below shows sample resistance and electrical conductivity of electrodes printed with a uniform thickness of different inks: pure carbon ink (Carbon); GiPEC 10% ink (GiPEC 10%); and GiPEC 20% ink (GiPEC 20%).

TABLE 1

| Sample | Sheet Resistance (Ω sq⁻¹) | | | Electrical Conductivity (S m⁻¹) | | |
|---|---|---|---|---|---|---|
| No. | Carbon | GiPEC 10% | GiPEC 20% | Carbon | GiPEC 10% | GiPEC 20% |
| 1 | 88.47 ± 0.85 | 95.49 ± 1.81 | 138.94 ± 4.77 | 1614.85 ± 15.51 | 1496.45 ± 28.33 | 1028.99 ± 34.70 |
| 2 | 88.20 ± 2.47 | 97.66 ± 1.29 | 139.38 ± 2.5 | 1620.50 ± 46.13 | 1462.92 ± 19.31 | 1025.19 ± 18.46 |
| 3 | 86.48 ± 0.97 | 96.20 ± 2.60 | 137.47 ± 3.9 | 1652.05 ± 18.64 | 1485.67 ± 39.74 | 1039.72 ± 29.45 |
| 4 | 88.75 ± 0.53 | 94.34 ± 4.12 | 137.91 ± 3.83 | 1609.76 ± 9.62 | 1516.21 ± 66.34 | 1036.38 ± 29.04 |
| Total | 87.97 ± 1.52 | 95.92 ± 2.61 | 138.42 ± 3.37 | 1624.28 ± 41.29 | 1490.31 ± 41.29 | 1032.57 ± 25.06 |
| Ave. | | | | | | |

In some embodiments, the surface of the finished SPE 202 may be modified with functional groups prior to antibody immobilization. For example, the SPE 202 may be treated with one or more of APTES (#440140, Sigma, USA), NHS (#130672, Sigma, USA) or EDC (#25952-53-8, TCI AMERICA, USA). However, the applicant found that the untreated SPE 202 performed equivalent or better in antibody immobilization than SPEs that are treated with the aforesaid functional groups.

In some embodiments, the finished SPE 202 is immobilized with a specific antibody, aptamer, DNA, or nanobody, depending on the target antigen the SPE is designed to detect. For example, an SPE that is configured to detect the presence of GFAP protein in a sample solution, the finished SPE can be immobilized with 10 μg mL⁻¹ GFAP antibody (#G3893, Monoclonal Anti-Glial Fibrillary GFAP antibody, Sigma, USA) by drop-casting the aliquoted concentration of the antibody on the working electrode 248. In some embodiments, to complete the interaction of the antibody with the electrode's surface, the SPE that is drop-casted with antibody is incubated for about 60 min in room temperature, followed by incubation in about 4° C. overnight.

In some embodiments, to prevent non-specific binding on the SPE 202, the surface of the working electrode 248 is covered e.g., with 0.005% (m/v) Bovine Serum Albumin (BSA), e.g., #sc-2323, ChemCruz, USA, and rinsed prior to interaction with a sample solution. For example, a prepared BSA solution is drop-casted on to the working electrode 248, and then the SPE is incubated for 20 min at room temperature and rinsed and dried prior to the final biosensing step. The introduction of the surface-blocking BSA to the working electrode 248 results in a higher charge transfer resistance.

In some embodiments, optimal immunoreaction occurs within 10-30 min incubation of the target antigen (e.g. 30 min for the GFAP protein) and the corresponding antibody (e.g. the GFAP antibody), measured in the presence of a 2.5 mM Fe(CN)₆^{3-/4-} redox probe.

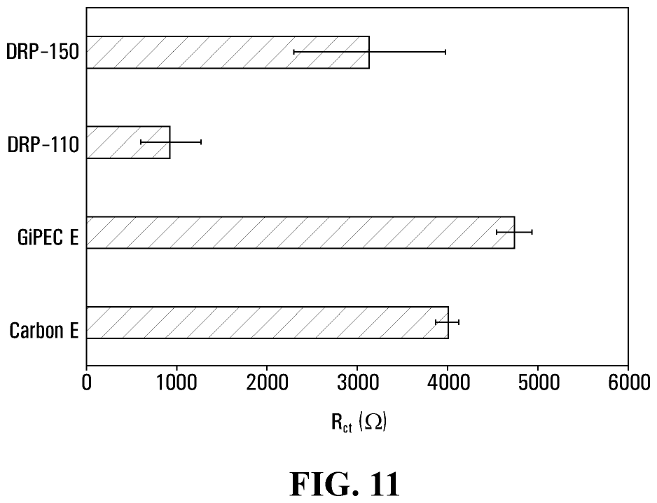

FIG. 11 shows a graph comparing the reproducibility of the GiPEC SPE (GiPEC E) with the respective reproducibility of the pure carbon electrode (Carbon E) and DropSens SPEs (DRP-150 and DRP-110). As shown in FIG. 11, the GiPEC SPE has a high reproducibility than the other electrodes. The error bars in FIG. 11 represent the standard deviation of the measurements.

In some embodiments, the SPE 202 may be used in the absence of the microfluidic unit of the sensor module 203. For example, a samples solution containing the target antigen (e.g. the GFAP protein) can be drop-casted on to the sensor portion 248b of the working electrode followed by incubation, washing, and redox probe addition steps prior to measuring the electrochemical properties using Electrochemical Impedimetric Spectroscopy (EIS). The microfluidic unit described herein is configured to automate the steps of samples introduction on to the working electrode 248, incubation within a defined period, washing for removing non-specific binding, and redox probe introduction on to the SPE.

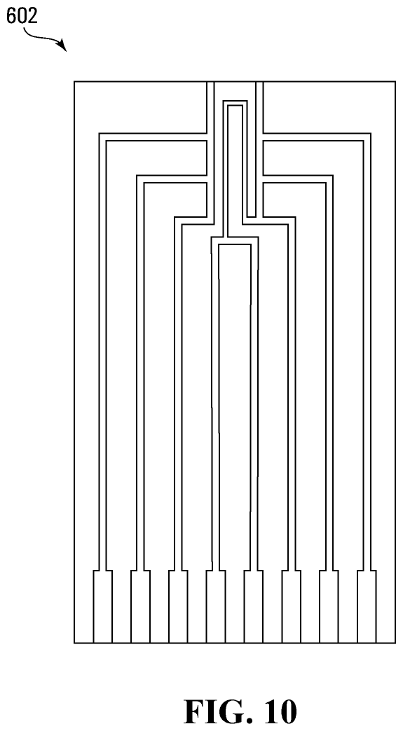

The applicant found that the aforesaid intermixed ink is viscous enough to print a nine-electrode SPE having a minimum feature size of about 300 μm. FIG. 10 shows a sample nine-electrode SPE 602 according to some embodiments. The SPE 602 functions similar to the SPE 202 described above with respect to FIGS. 7 and 8, and may be made of the same or similar materials via the same or a similar process as those described with respect to SPE 202.

Referring back to FIG. 7, the microfluidic unit according to some embodiments has a plurality of layers 206 to 222, defining a plurality of compartments: an inlet reservoir 252 in the form of a hole for receiving a samples solution (e.g. a biosample collected from a patient); a side redox route 254 wherein the presoaked redox reagents mix with the samples solution; and a biosensing chamber 256 wherein the antibody-antigen interaction occurs on top of the SPE 202. In some embodiments, one or more of the layers are made of sheets of Poly(methyl methacrylate) (PMMA) or pressure sensitive adhesive (PSA). The layers 214, 218, and 222 may have the same or different thicknesses. In the illustrated embodiment, layers 206, 208, 212, 216, and 220 are pressure sensitive adhesive PSA layers for bonding layers 204, 214, 218, and 222. PSA was also used to make thin fluid channel network. In some implementations, the layers 212, 214, 216, 218, and 220 are PSA and PMMA layers for redox mixing channel. In some embodiments, one or more of the inlet reservoirs 252, the side redox route 254, and the biosensing chamber 256 are formed by laser cutting, for example by a CO₂ laser. The microfluidic unit also has sheets 250a, 250b,250c for controlling the capillary flow of a sample solution at the different compartments and microfluidic channels of the microfluidic unit. In some embodiments, one or more of the sheets 250a,250b,250c comprise porous fibers (e.g. virgin wood fiber tissue wipers or nitrocellulose (NC)) that are patterned by laser cutting and contribute to the flow distribution to different channels.

Layer 206 is the lowermost layer of the microfluidic unit of the sensor module 203. Layer 206 is coupled to the top surface of the bottom substrate layer 205. In some implementations, the layer 206 is a PSA layer and is provided as an adhesive. Layer 206 has a plurality of electrode openings 226, which may be formed by laser cutting, corresponding to the sensor portions 244b,246b,248b of the SPE 202 such that the sensors portions are exposed through the electrode openings 226 to the biosensing chamber 256. Layer 206 is configured to electrically isolate the conductor lead portions 244a,246a,248a from the sensor portions 244b,246b,248b of the SPE 202.

Layer 208 is coupled to the top surface of layer 206. In some implementations, the layer 208 is a PSA layer for entrance and vent microchannels. The microfluidic sheet 250a is placed on layer 208 and is configured to provide microfluidic channels to control the flow of a sample solution to the working electrode 248b of the SPE 202. In some embodiments, the microfluidic sheet 250a is an NC-coated PET sheet that is laser cut to form microfluidic channels. In some embodiments, each microchannel has a channel width of about 100 μm to 1 mm, which eliminates the need for microfabrication technology to manufacture the microfluidic unit. In contrast, prior art microfluidic circuits, such as those described in U.S. Pat. No. 9,822,890, entitled “METHOD AND SYSTEM FOR PRE-PROGRAMMED SELF-POWER MICROFLUIDIC CIRCUITS”, issued on Nov. 21, 2017, are made by complex and time-consuming microfabrication, lithography, molding, and hot-embossing methods and, consequently, may take weeks to months to fabricate or modify. By eliminating the need for microfabrication, the manufacturing time for the microfluidic unit may be reduced to days or hours without losing the performance or reproducibility.

The capillary sheet 250b is placed on top of the working electrode 248b of SPE 202 to control the capillary flow of the sample solution from the inlet reservoir 252 and direct the solution to spread over only the working electrode 248b to initiate immunoreaction while preventing the overflow of the solution to the other two electrodes 244b, 246b. In some embodiments, the capillary sheet 250b comprises pattered wood fiber. The capillary sheet 250b is in direct contact with the microfluidic sheet 250a and layer 208, thereby preventing extra accumulation of the sample solution on the working electrode 248b. While a small volume of the sample solution starts incubating on the SPE 202, the remaining volume of the sample solution flows to the side redox route 254 to control redox mixing and washing steps as the next steps of biosensing.

In some embodiment, the capillary sheet 250b maybe mounted underneath a porous membrane (made of e.g. parylene or cellulose; porosity: 200 nm-20 um) to filter bioparticles (e.g. cells, viruses, bacteria, and exosomes) prior to conducting the sample to the sensor zone 256 via the capillary sheet 250b. The capillary sheet in this configuration (placed underneath the porous membrane) is able to draw the liquid through the pores using self-powered capillary force without a need for external pressure on top of the membrane to push the liquid through the pores.

In the illustrated embodiment, the side redox route 254 defined in layers 212 to 218 contains a zigzag channel 262, a redox mixing chamber 264, and a delay channel 266. The redox sheet 250c is placed inside the redox mixing chamber 264. In some embodiments, the redox sheet 250c is an NC-coated sheet that is laser cut, soaked in a high concentration of the redox solution (e.g. 10-100 mM solution of Fe(CN)₆^{3-/4-}) and dried in room temperature, prior to placement in chamber 264. The zigzag, conical, or spiral channel 262 at the entrance of the side redox route 254 provides resistance and damping against possible abrupt introduction of the samples solution at the inlet, controls the flow rate and volume of the samples solution conducted through the redox probe route 262, and increases reproducibility of the redox mixing and washing steps. A combined bump-shape projection at the redox mixing chamber (at the entrance of the delay channel 266), a step-like feature designed at the exit of the delay channel, and a hydrophobic treatment of the PMMA or PSA walls of the delay channel function as a tunable stop valve. The tunable stop valve is configured to provide sufficient time for dissolving and homogenous diffusion of the NC-soaked redox probes into the samples solution and to delay the delivery of the dissolved redox probe into the biosensing chamber 256. This delay provides the “time span” needed for the completion of the immuno-reaction on the SPE 202. In some embodiments, the time span ranges from about 25±5 min GFAP biosensing. Knowing that all channels' sizes are larger than 300 um, the alignment of the layers even manually creates minimal misalignment, therefore the applicant reports the high performance reproducibility of the intra variation of delay/stop valves (<1%), making the entire microfluidic chip performing highly reproducible without need for using expensive device for alignment of microfluidic layers

Upon an automatic opening the stop valve, the redox probe is delivered into the sensing chamber 256, covers the three electrodes 244b,246b,248b, washes non-specific binding molecules, and provides a defined concentration of the redox buffer on the electrodes 244b,246b,248b needed for accurate electrochemical biosensing. The electrochemical signals of the electrodes 244a,246a,248a can be recorded sometime (30 seconds to 4 min) after opening the stop valve. For example, for GFAP biosensing, the electrochemical signals can be measured about 2-3 min after the opening of the stop valve, such that the entire process of biosensing GFAP from sample to result takes about 30 min. The waiting period between the opening of the stop valve and the recording of electrochemical signals is tunable depending upon the incubation time involved. The entire process of sample-to-result can be tubed to be within 5-60 min depending upon the type of molecules of interest to detect.

Layer 222 is the uppermost layer of the sensor module 203 and is configured to provide open access to the inlet reservoir 252 and unobstructed viewing of the biosensing chamber 256. In some implementations, the layer 222 is an upper layer formed with PMMA and is provided for inlet reservoir. Layer 220 bonds layer 222 to layer 218. In some implementations, the layer 220 is a PSA layer and is provided as an adhesive.

In operation, a samples solution is introduced into the inlet reservoir 252 at layer 222. Some of the sample solution flows on to the working electrode 248b via the microfluidic channels in layer 208 and the remainder flows to the side redox route 254 defined in layers 212 to 218. In the side redox route 254, the samples solution first flows into the redox mixing chamber 264 where it is mixed with redox to produce a redox-mixed sample. The stop valve delays the delivery of the redox-mixed sample into the biosensing chamber 256. After a predetermined time span has lapsed, the redox-mixed sample enters the biosensing chamber 256 and comes into contact with the electrodes 244b,246b,248b of the SPE 202. After a predetermined waiting period from the entry of the redox-mixed sample into the biosensing chamber 256, electrochemical signals of the sensor module 203 are recorded via the SPE 202. To record the electrochemical signals, the SPE 202 is connected at the conductor lead portions 244a,246a,248a to a portable or handheld potentiostat of the reader module by a standard electrode cable in some embodiments. Using the sensor unit and the microfluidic unit of the sensor module 203, the concentrations of a target molecule, such as GFAP antigen, in spiked or clinical samples can be quantified by subtracting the electrical signals detected before and after immunoreaction.

In some embodiments, the layers 204 to 222 are made of flexible materials so that the sensor module 203 may be incorporated into wearable biosensing systems, for example as a wearable biosensor mounted on the skin for measuring the biomarkers of stress.

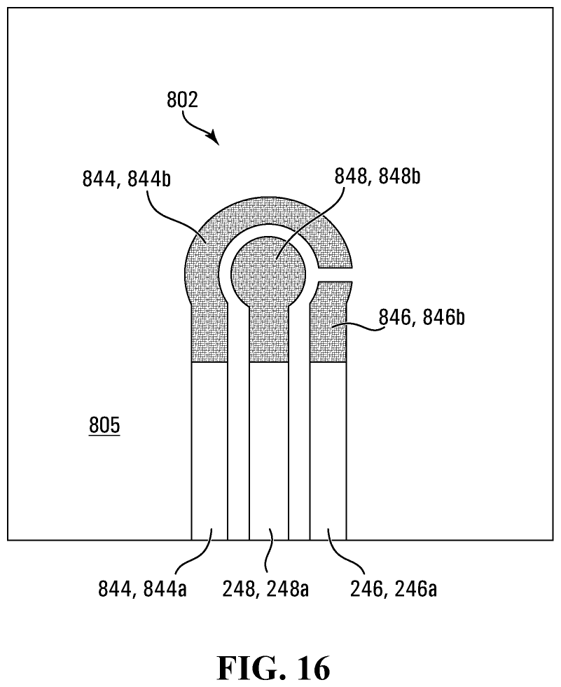

FIG. 16 shows an SPE 802 that can be used to detect the presence and concentration of proteins (e.g. GFAP, UCHL-1, viral spiked and nucleocapsid proteins) in a sample solution. The SPE 802 has three electrodes: a reference electrode (RE) 844, a control electrode (CE) 846, and a working electrode (WE) 848. The RE 844, CE 846, and WE 848 each has a respective conductor lead portion 844a,846a,848a, and a respective sensor portion 844b,846b,848b. In some embodiments, the SPE 802 is printed on a substrate 805 which may be the same as or similar to substrate 205 described above with respect to FIG. 8.

In some embodiments, the RE 844, CE 846, and WE 848 are nano-porous carbon electrodes, wherein WE 848 are functionalized with hydroxylamine (NH₂OH) to increase conductivity and to facilitate binding of a linker (e.g. carboxylic linkers) on the surface of the electrodes. The SPE 802 is then modified with nitroso functional group to immobilize GFAP antibody.

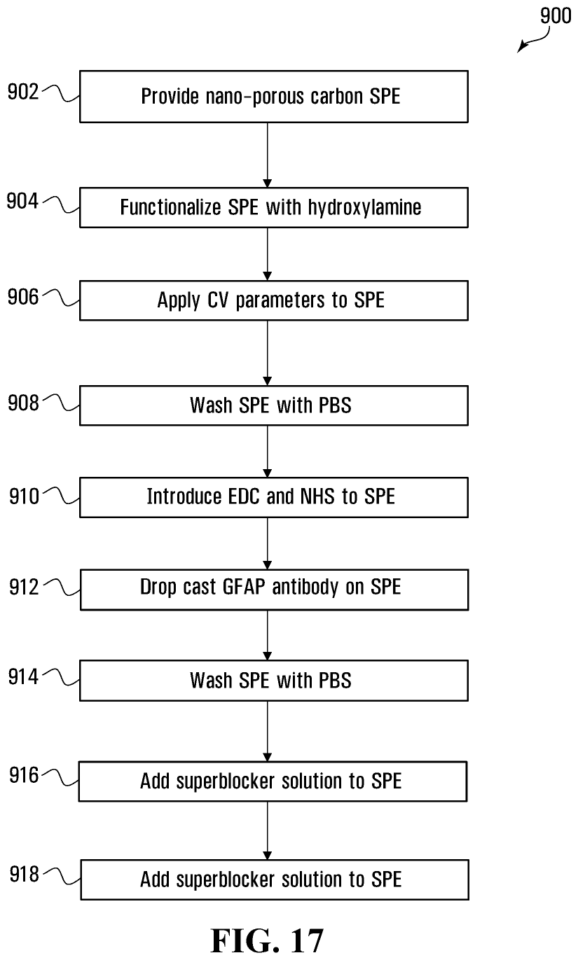

FIG. 17 shows a sample process 900 for making SPE 802. The process 900 starts at step 902 where the nano-porous carbon SPE 802 is provided. At step 904, the WE 848 of SPE 802 is functionalized with hydroxylamine. In one example, about 140 μL of 0.15 M hydroxylamine is added on the WE 848 of SPE 802.

At step 906, cyclic voltammetry (CV) parameters are applied to the SPE 802, for example a potential of 1 V to −1.5 V, and 10 scans at a scan rate of 50 mV/s, to oxidize the hydroxylamine and create nitroso groups on the SPE 802.

At step 908, the SPE 802 is washed with PBS.

At step 910, 60 mM of 1-ethyl-3-(3-dimethylaminopropyl) carbodiimide (EDC) and 80 mM of N-hydroxysuccinimide (NHS) (1:1) are introduced to the SPE 802 for 4 hours. Carboxylic linkers are created on the SPE 802 to enable binding of the GFAP antibody to the electrode surface.

At step 912, GFAP antibody is drop-casted on the SPE 802. In one example, 7 μL of GFAP antibody (10 μg/mL in PBS, pH 7.0/7.4) is added to and incubated on the electrode surface of SPE 802 at 4° C. overnight.

At step 914, the SPE 802 is washed with PBS to remove any unbound antibodies.

At step 916, 50% superblocker solution (e.g. 4 μL) is added on the SPE 802 and the SPE 802 is incubated for 5 min to prevent non-specific binding on the SPE during the interaction of the SPE with a test sample.

At step 918, the SPE 802 is cleaned again with PBS to remove any free and unbound molecules of the superblocker solution.

The applicant used atomic force microscopy (AFM) in a tapping mode to visualize the morphological change occurring during the surface modification of the SPE 802. The applicant found that the addition of the GFAP antibody on the SPE 802 caused a change in surface morphology of the SPE. Distinct globular structures associated with the GFAP antibody appeared on SPE 802, thereby increasing the surface area of the SPE 802.

The SPE 802 can be calibrated by spiking samples from healthy donors with GFAP protein in phosphate buffer saline (PBS). GFAP binding frequency is translated to the concentration levels of the spiked samples using electrochemical impedance spectroscopy (EIS).

In operation, a samples solution (e.g. about 5 μL) is added to the SPE 802 and incubated for about 30 min. The SPE 802 is then washed with PBS to clean residues and unbound GFAP proteins from the SPE. In some embodiments, the GFAP protein concentration on the SPE 802 can be electrochemically detected by adjusting EIS parameters, including for example the potential 0.17 V, 10 mV amplitude of the applied AC signal, and scanning frequency range 800 to 0.05 Hz in the presence of 140 μL of 2.5 mM Fe(CN)₆^{3-/4-} redox probe solution prepared in 10 mM PBS (pH/7.4).

The SPE 802 can be used, for example in a point-of-care system, to rapidly (e.g. about 30 min) detect the presence and concentration of GFAP protein in a patient sample after neural injury experienced by the patient, including traumatic brain injury, spinal cord injury, and stroke.

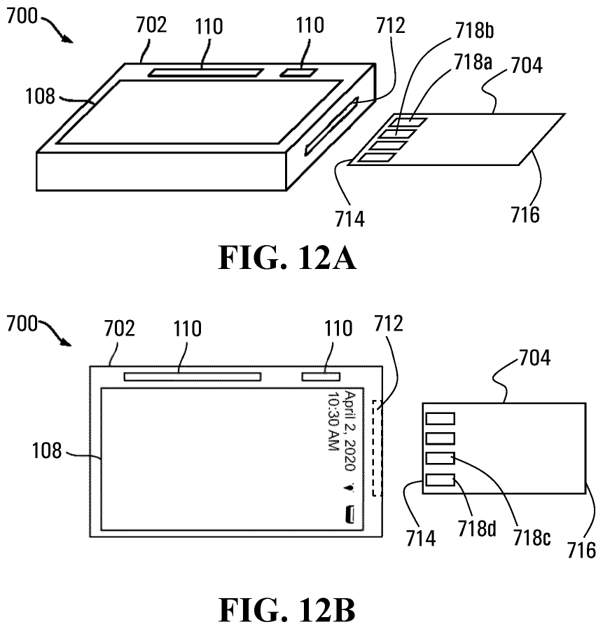

Turning now to FIGS. 12A and 12B, an alternative electrochemical-sensor system 700 is shown, which may be used for analyzing, determining, and monitoring the presence of one or more target antigens in a sample. Like system 100 described above with respect to FIGS. 1A and 1B, system 700 is configured to be portable and may be used for home-based testing for disease diagnosis and prognosis.

The electrochemical-sensor system 700 in some embodiments comprises a reader module 702 in the form of a point-of-care (PoC) device which may be sized for portability for personal use and a sensor module comprising a sensor unit 704 which may be disposable. The sensor module of system 700 may comprise a microfluidic unit (not shown), which may be the same as or similar to either of the microfluidic units described above.

In the illustrated embodiment, the reader module 702 comprises a user interface, which may include an output mechanism such as a display or touch screen 108 and an input mechanism such as touch screen 108 and/or one or more buttons 110 for receiving user input, the same as or similar to the like-numbered components described above with respect to system 100. The reader module 702 also comprises a receiving port 712 for receiving a proximal side 714 of the sensor unit 704, a control structure (not shown) comprising one or more controllers, and relevant circuitries. In some embodiments, the control structure comprises one or more master microcontrollers and one or more slave microcontrollers, each of the latter being in communication with at least one of the master microcontrollers. The reader module 702 may comprise a processor, memory, data storage, software, hardware, and/or firmware known to those skilled in the art. In some embodiments, the reader module and/or the control structure thereof is configured to enable data storage in an online repository (e.g. cloud storage), instead of on the device itself, which aids in storage capacity and ease of subsequent access.

The reader module 702 comprises a power source (not shown) such as a battery for powering various components thereof. In sample embodiments, a commercial battery pack providing over 16,750 mAh may be used as a portable source of power for the reader module 702. The power source may be rechargeable using, for example, a USB port, thereby simplifying the on-board power management of the reader module.

The circuitries of the reader module 702 may include an analysis circuitry such as a potentiostat circuitry for biosensing and a monitoring circuitry for other tasks such as performing user-instructed operations, detecting the insertion of the sensor unit 704, reading and displaying the measured levels of target antigens, storing measurement data, transmitting measurement data to a remote device for trend tracking, and/or the like.

While the reader module 702 is described hereinbelow in association with sensor unit 704, it can be appreciated that the reader module 702 may be configured to operate with sensor module 103,203 and/or SPE 202,602, described above. Similarly, in alternative embodiments, the sensor unit 704 may operate with the reader module 102 and/or a microfluidic unit such as those described above.

In some embodiments, the system 700 is configured to simultaneously detect multiple target biomolecules or bioparticles (“multiplexing”). In some embodiments, the reader module 702 is configured to simultaneously detect up to 8 target biomolecules/bioparticles. In some embodiments, the reader module 702 displays results in analog and/or digital formats. In some embodiments, the data acquired by the reader module 702 may be stored wirelessly in a cloud server for further investigation and statistical analysis. The multiplexing capability of the system 700, may be useful in detecting and quantifying, for example Cleaved-Tau Protein (C-Tau) and Neuron-Filament (NFL) proteins in the blood of traumatic brain injury (TBI) patients. For example, using system 700, the applicant was able to selectively detect the C-Tau and NFL proteins in blood samples in less than 30 min within the dynamic range of 10 pg/mL-100 ng/ml and the sensitivity range of 47 μA/pg mm²-65 μA/pg mm².

In some embodiments, the reader module 702 uses differential pulse voltammetry (DPV). DPV is a process by which electrochemical measurements are made. The DPV process involves generating a ramped voltage step function across the sample in question and stepping up the voltage in set increments over a fixed period where the resultant current is measured. The DPV process characterizes a redox reaction, from which the concentration of a desired antigen can be determined. Given its ability to cancel the background signal, DPV offers high sensitivity to potential and current, thus enabling the detection of electroactive species at low concentrations. DPV current response decreases for the increased antigen concentration, resulting from the inactive antigen-antibody complex on the sensor unit 704. This implementation helps increase the measurable range of analytes down to parts per billion in complex bodily fluids like the blood.

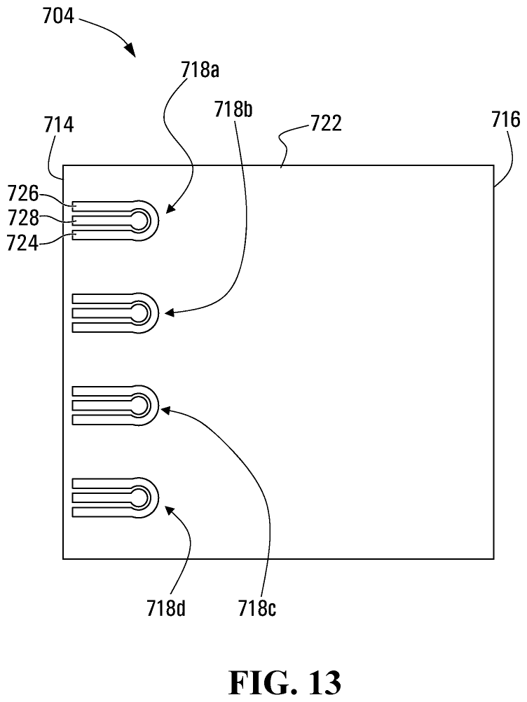

In the illustrated embodiment, as shown in FIGS. 12A, 12B, and 13, the sensor unit 704 comprises a substrate 722 in the form of a sheet having some thickness and one or more sensor strips 718a,718b,718c,718d positioned on the upper surface of the substrate 722. In some embodiments, the substrate 722 is made of glass. Each of the sensor strips 718a,718b,718c,718d has a respective RE 724, a respective CE 726, and a respective WE 728 adjacent the proximal side 714. While the sensor unit 704 in the illustrated embodiment has four sensor strips, it can be appreciated that the sensor unit 704 may have fewer or more sensor strips in other embodiments.

In some embodiments, each sensor strip 718a, 718b,718c, 718d is fabricated by sputtering the conducting material on the substrate 722 and using shadow masks (not shown). The shadow masks may be made of thin PMMA. Prior to sputtering, the substrate 722 may be cleaned by an isopropyl alcohol rinse followed by an ultrasonication step for a predetermined period of time in deionized (DI) water. The shadow masks may be prepared using a laser cutter (e.g., Universal VLS3.60) and the shadow masks are used to feature the sensor strips on the substrate 722. A pattern may be engraved in the shadow masks to help align the substrate with respect to the sensor strips and enable consistent fabrication of the sensor strips. The substrate is then mounted on to the engraved pattern on a first mask of the shadow masks and taped down, using for example a Kapton tape. A sputtering system (e.g. Kurt J. Lesker CMS-18 multi-source sputtering system) is used to sputter the sensor strips on the substrate 722. In sample embodiments, a 2-minute cycle at 300 W of titanium is sputtered to act like a 20 nm adhesion layer for the WEs 728 and CEs 726, and then a 3-minute cycle at 300 W of gold (or chrome) is sputtered to deposit a 150 nm layer on top of the titanium-coated WEs 728 and CEs 726.

After the sputtering the WEs 728 and CEs 726, the substrate 722 is then placed underneath a second mask of the shadow masks, lined up with the engraved pattern, and taped down to sputter the REs 724. In sample embodiments, a 2-minute cycle at 300 W of titanium is sputtered to act like a 20 nm adhesion layer for the REs 724, and then a 3-minute cycle at 300 W of platinum is sputtered to deposit a 120 nm layer on top of the titanium-coated the REs 724. In some embodiments, the sputtered CEs, REs, WEs are flame-annealed in a vacuum oven at about 482° F. for about 12 hrs to stabilize the sputtered materials process and complete the fabrication of sensor strips 718a, 718b,718c,718d.

In some embodiments, the sensor strips 718a,718b,718c, 718d are functionalized by self-assembled monolayer (SAM) modification of the gold WEs 728 and CEs 726. In sample embodiments, the sensor strips are immersed in 5 mM 11-MUA in an ethanoic solution for about 24 hrs in the presence of nitrogen, resulting in the formation of alkanethiol on the gold WEs 728 and CEs 726. The sensor strips are then washed with ethanol to remove unbound molecules. The carboxylate group of SAM is used to immobilize the antibody over the gold WEs 728 and CEs 726. Carboxylic acid terminal of 11-MUA is activated by immersing the sensor strips in a mixture of water-soluble carbodiimide (50 mM) and N-Hydrosuccinimide (NHS) (50 mM) for about 2 hrs. The activation of the carboxylic group on surface of the sensor strips allowed for binding of the antibodies to the surface of the gold WEs 728 and CEs 726 via amine functional groups, which enables for example the detection of human NFL and C-Tau analytes using the electrochemical DPV technique described in detail below.

In sample embodiments, 20 μL of an antibody solution (e.g., 25 μg/mL in 10 mM PBS, pH 7.0) is drop casted on the working electrode of the sensor strips using, for example, an established pipetting method. Antibodies are incubated on the functionalized sensor strips 718a,718b,718c,718d in water-saturated atmosphere for about 45 min at room temperature. The WEs 728 are then blocked with incubating 0.25% BSA for about 30 min to prevent non-specific binding. In some embodiments, after each step of the surface modification with SAM, antibody coating, and surface blocking, the sensor strips are rinsed three times with phosphate-buffered saline (PBS).

In sample embodiments, target antigens (e.g., NFL and C-Tau proteins) can be detected by adding 20 μL of a spiked sample prepared in PBS or a serum or clinical samples on to the SAM modified electrode. The target antigens are allowed for binding with their respective antibodies by incubating them for about 30 min in water saturated atmosphere. After the incubation, the SAM modified sensor strips are washed with PBS to remove the unbound targets and residues from the sample matrix. Electrochemical measurements of the sensor strips 718a, 718b,718c,718d can be performed by the reader module 702 or a commercial potentiostat using DPV, in the presence of 4 mM [K(Fe(CN)₆]^{3-/4-} redox probe.

While the sensor strips 718a, 718b,718c,718d are described in the absence of a microfluidic unit, it can be appreciated that each sensor strip may operate with a respective microfluidic unit or all the sensor strips may operate with a single microfluidic unit, the microfluidic unit (such as those described above) being configured to receive and prepare a samples solution prior to delivering same to the WEs 728 of each of the sensor strips.

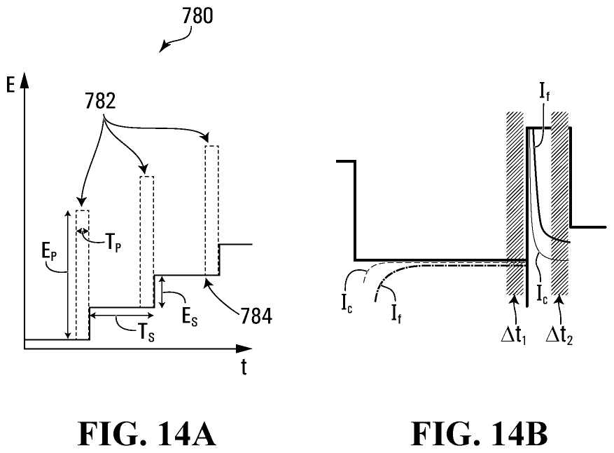

As discussed above, the reader module uses DPV. FIG. 14A shows a typical applied DPV voltage waveform 780 and FIG. 14B shows the resulting current from the DPV voltage. DPV consists of a raising base potential with short time and high potential pulses 782. As shown in FIG. 14A, the waveform 780 applied to the electrodes of the sensor strips is a pulse signal superimposed on to a base ramp 784. The waveform 780 is characterized by the initial potential, step time T_{S}, step potential E_{S}, pulse time T_{P}, and pulse potential E_{P}. The resulting current consists of two components: faradaic current from charging the cell (I_{f}) and reaction current (I_{C}). The electrical current response is measured twice in each pulse time T_{P}, first before the start of the pulse, and second at the end of the pulse signal (i.e., Δt1 and Δt2 in FIG. 14B). Taking the difference of the currents before the pulse at Δt₁ and at the end of the pulse at Δt₂ reduces the effect of the faradaic current and thus more accurately measures the reaction current. Subsequently, the current difference can be plotted against the base ramp potential applied for recording the signal. When this process is repeated for a range of antigen concentrations, a calibration curve can be derived from the resulting waveform.

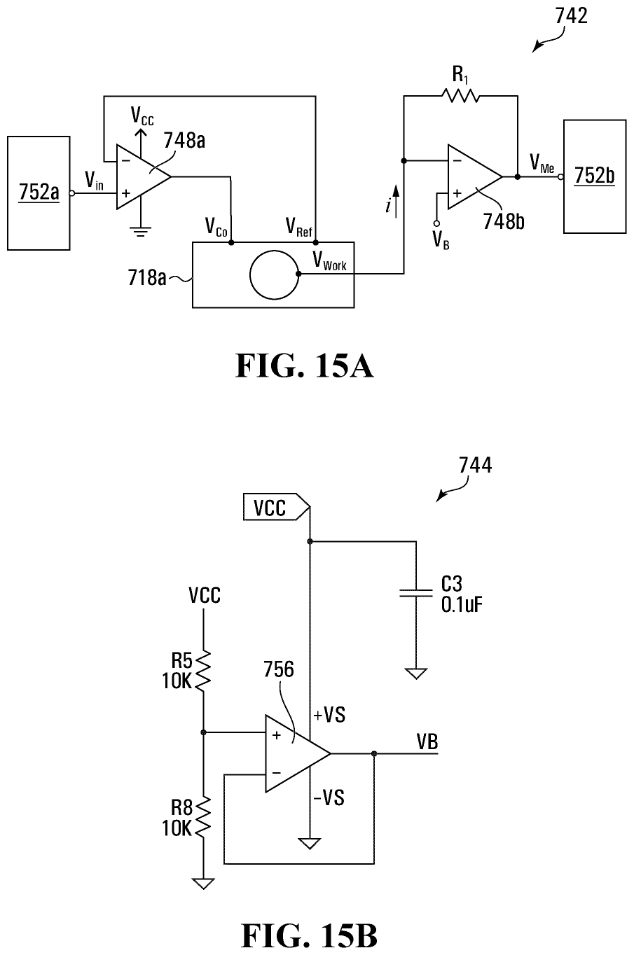

With reference to FIGS. 15A and 15B, the reader module 702 comprises a plurality of circuits 742 and 744 for electrically engaging the electrodes 724 to 728 when the proximal side 714 of the sensor unit 704 is inserted into the receiving port 712.

FIG. 15A shows a sample potentiostat circuit 742 comprising two op-amps 748a, 748b configured to connect to the electrodes 724 to 728 of one of the sensor strips (e.g. 718a), when the proximal side 714 of the sensor unit 704 is inserted into the receiving port 712. The first op-amp 748a takes the ramped signal input signal V_{IN} from a master microcontroller 752a (e.g. Raspberry Pi Zero W) of the control structure of the reader module 702 and forces the potential V_{Ref} at the RE 724 to match V_{IN}. A potential is applied between the CE 726 (V_{Co}) and WE 728 (V_{Work}), setting the redox potential up for the reaction, which generates an electrical current. V_{Work} is held constant at a bias voltage V_{B}. The reaction current then passes through the resistor R₁, generating a voltage, V_{Me}, which is measured using a slave microcontroller 752b of the control structure of the reader module 702. The master microcontroller 752a then calculates the electrical current based on Equation 1:

$$\\ \begin{matrix}i = ( - {V}_{MEAS} + {V}_{B} ) / R 1 & ( Eq . 1 )\end{matrix}$$

The bias voltage is set to the middle of the input voltage range (e.g. 1.65 V) using a buffered resistor voltage divider, an example of which is shown as circuit 744 in FIG. 15B, which allows for the voltage seen across the RE 724 and WE 728 to be bipolar (i.e., where the voltage of 0 to 3.3 V is applied to the RE, the cell voltage is −1.65 to +1.65 V). Circuit 744 comprises an op-amp 756 which may be selected to have a high common-mode rejection ratio (e.g. 85 dB) and input voltage noise of about 40 nV/√Hz to limit the noise in the amplified signals. In some embodiments, the low input bias current (e.g. 10 nA) minimizes the effect of op-amp bias current on the measured current.

In some embodiments, the slave microcontroller 752b is configured to run the DPV test for one or more sensor strips. The slave microcontroller 752b controls the potentiostat circuit 742 to apply a voltage and the slave microcontroller 752b converts the resulting response into a digital signal. In a sample embodiment, the slave microcontroller is a SAML21E microcontroller having one or more 12-bit digital to analog converters (DACs), which allow for ˜1 mV resolution in applied reference voltages. The slave microcontroller 752b also has a 12-bit analog to digital converter (ADC) that allows for ˜1 mV accuracy in voltage measurements. The slave microcontroller 752b then communicates the digital signal to the master microcontroller 752b. In some embodiments, the master microcontroller 752a and the slave microcontroller 752b communicate with one another via a serial peripheral interface.

In a sample embodiment, the DPV parameters for the reader module 702 are listed in Table 2 below.

TABLE 2

| DPV Parameter | Value |
|---|---|
| Initial Potential (mV) | −500 |
| Final Potential (mV) | 500 |
| Step Potential (mV) | 5 |
| Pulse Width (mS) | 100 |
| Pulse Period (mS) | 200 |
| Pulse Amplitude (mV) | 30 |

Prior to use, the sensor strips 718a, 718b, 718c, 718d are each functionalized as described above with a respective antibody each configured to bind with a specific antigen. In operation, a sample solution containing at least one target antigen binds to the respective antibodies on the sensor strips. The sensor strips, when inserted into the reader module 702, initiates a DPV signal, which triggers a redox reaction. The reader module 702 measures the redox current of each of the sensor strips through the DPV technique. In some embodiments, the electrochemical reaction, initiated by the DPV, produces an electrical current through the CE 726. The reader module 702 measures the current between the CE 726 and WE 728 and correlates the measure current with the target antigen concentration. The reader module 702 can simultaneously measure the currents in multiple sensor strips in parallel. In some embodiments, the DPV signal is displayed on the reader module 702, and the corresponding data may be stored in a cloud-based data storage module.

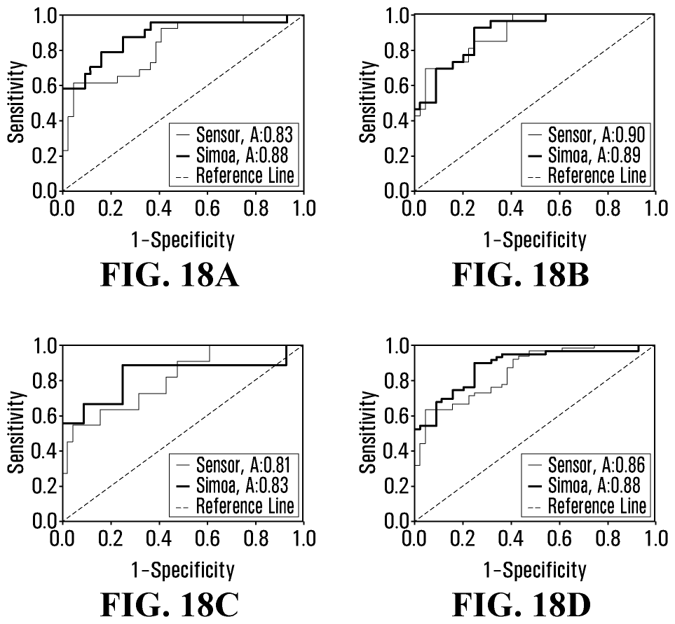

In FIGS. 18A to 18D, it has been demonstrated that the GFAP sensor module 203 operated in a dynamic range of 100 fg/mL-10 ng/ml, offering the limit of detection of 86.6 fg/mL and the sensitivity of 20.3 ΩmL/pg mm2 in the serum. The GFAP sensor module 203 could accurately and rapidly quantified GFAP in the serum with a demonstrated clinical accuracy equivalent to the most sensitive ELISA assay (SIMOA) for diagnosis and prognosis of TBI, stroke and SCI. The clinical sensitivity and specificity of the GFAP sensor module 203 were evaluated using the receiver operating characteristic (ROC) curve. ROC analysis was used to identify whether the sensor system 203 detects GFAP concentration above or below injury threshold concentrations of 40 pg/mL for TBI, 100 pg/mL for stroke, and 50 pg/mL for SCI, and 75 pg/mL when all the clinical samples were assessed together (n=107) (FIGS. 18A to 18D). These thresholds were set based on reported thresholds distinguishing clinically injured patients or those with stroke and healthy subjects. The clinical sensitivity and specificity of the GFAP sensor module 203 for the detection of 70 samples (TBI: 26, control: 44) were 84.62% (CI, 65.1% to 95.6%) and 61.36% (CI, 45.5% to 75.6%), respectively. In contrast, SIMOA recorded 95.83% sensitivity (CI, 78.9% to 99.9%) and 34.09% specificity (CI, 20.5% to 49.9%) (FIG. 18A). The accuracy of GFAP measurement was calculated to be 70% and 38.6% for the sensor module 203 and the SIMOA assay, respectively. The area under the curve (AUC) for the TBI samples measured with the sensor module and SIMOA were 83.51% (binomial exact CI, 0.72 to 0.90) and 87.84% (binomial exact CI, 0.785 to 0.950), respectively. The results show that the GFAP sensor module was more likely to detect true negative cases better than the SIMOA assay.

In some embodiments, the GFAP sensor module 203 was successfully predictive for point-of-care diagnosis and prognosis of brain stroke (FIG. 18B). 70 samples (stroke: 26, control: 44) were considered for the ROC analysis of stroke. The GFAP sensor module 1020 delivered clinical sensitivity of 73.08% (CI, 52.2% to 88.4%) and specificity of 77.27% (CI, 62.2%-88.5%), whereas the SIMOA assay delivered clinical sensitivity of 80.77% (CI, 60.6% to 93.4%) and specificity of 75% (CI, 59.7% to 86.8%) in the detection of GFAP in the blood of patients with stroke. The AUC and accuracy of GFAP concentration using the GFAP sensor module 203 were 81.4% and 87.84% (binomial exact CI, 0.777 to 0.944). In contrast, AUC and accuracy of GFAP concentration using the SIMOA was determined to be 77.94% and 89.63% (binomial exact CI, 0.800 to 0.956), respectively. The GFAP sensor module 203 offered a more accurate detection than SIMOA to detect stroke patients within the GFAP detection range of SIMOA.

In some embodiments, the sensor module 203 was successfully predictive for point-of-care diagnosis and prognosis of SCI patients (FIG. 18C). ROC analysis in SCI samples with GFAP was performed by considering 55 clinical samples (SCI: 11, Control: 44). The GFAP sensor module 1020 provided clinical sensitivity of 72.73% (CI, 39.0% to 94.0%) and specificity of 63.64% (CI, 47.8%-77.6%) for the detection of SCI patients. On the other hand, SIMOA provided clinical sensitivity of 88.89% (CI, 51.8% to 99.7%) and specificity of 34.09% (CI, 20.5%-49.9%). The GFAP sensor module 203 and SIMOA provided accuracies of 65.45% and 41.81%, respectively, and AUCs of 81.27% (binomial exact CI, 0.684 to 0.905) and AUCs of 83.11% (binomial exact CI, 0.703 to 0.920), respectively, for the detection of GFAP concentration in SCI samples. Based on these statistical analyses, we infer that the GFAP sensor module 203 provided more accurate detection for SCI injuries and distinguished more true negatives than SIMOA.

In some embodiments, the sensor module 203 prepared with the protocol of FIG. 17 was successfully predictive for point-of-care diagnosis and prognosis of patients with central nervous system (CNS) injuries (FIG. 18D). A threshold of 75 pg/mL was set to distinguish clinically abnormal GFAP concentration compared to healthy controls to compare the overall performance of the GFAP sensor module 203 and SIMOA. All the clinical samples (n=63; TBI: 26, stroke: 26, SCI: 11) and controls (n=44) were considered for this cumulative analysis. The clinical sensitivity and specificity of the GFAP sensor module 203 in detecting GFAP in clinical samples were calculated to be 71.43% (CI, 58.7% to 82.1%) and 72.73% (CI, 57.2%-85.0%), respectively. For the GFAP measured with SIMOA, the clinical sensitivity and specificity were calculated to be 89.83% (CI, 79.2% to 96.2%) and 75% (CI, 59.7%-86.8%), respectively. The GFAP sensor module 203 and SIMOA offered accuracies of 73.83% and 80.37%, respectively. The AUCs for each of the GFAP sensor module 203 and SIMOA were measured to be 84.63% (binomial exact CI, 0.763 to 0.908) and 88.23% (binomial exact CI, 0.803 to 0.937), respectively, for the detection of GFAP concentration in 107 clinical samples.

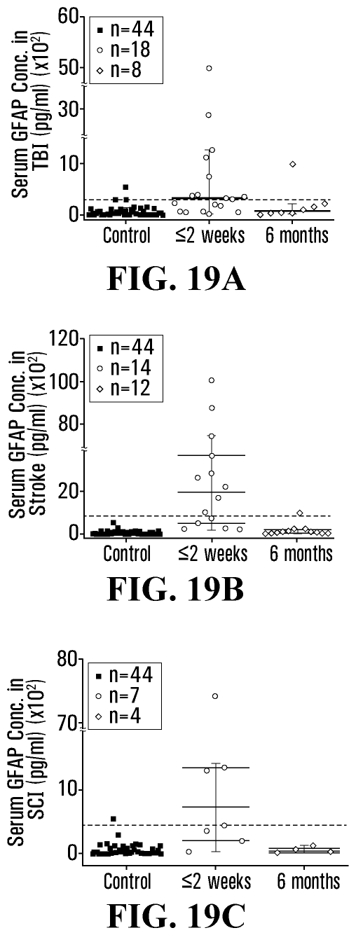

In some embodiments, the sensor module 203 prepared with the protocol presented in FIG. 17 was shown to be able to measure the change in GFAP concentrations over the course of weeks post-injury and could be used as a quantitative indication of patient recovery (FIGS. 19A to 19C). Clinical samples have been evaluated from the three CNS injury conditions (TBI, SCI, and stroke) obtained within ≤2-weeks for TBI and stroke and ≤3-weeks for SCI post-injury, and 6 months post-injury. Cut-off concentrations separating the injury state from the healthy state were manually set (horizontal dashed line) at 40 pg/mL for TBI (FIG. 19A), 100 pg/mL for stroke (FIG. 19B), and 50 pg/mL for SCI (FIG. 19C) based on the data in literature. From the clinical cohort measured, the GFAP concentration was recorded higher for the TBI clinical samples compared to 6 months (FIG. 19A). A similar trend was observed in the stroke (FIG. 19B), and SCI (FIG. 19C) samples based on their corresponding cut-off concentrations. The change in GFAP concentration peaking and normalizing overtime was uniquely detectable by the GFAP sensor module 203. Clinical adoption of the GFAP sensor module 203 can complement or replace existing diagnostic modalities such as imaging and questionnaires to enable rapid, accurate, cost-effective and near real-time point-of-care analysis in different clinical and remote settings.

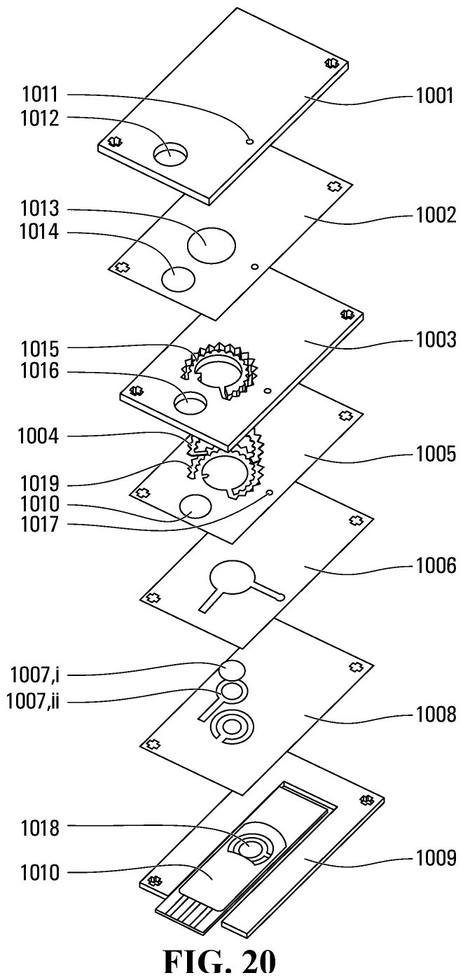

FIG. 20 is an exploded view of another novel sensor module 1020 that can be used in the electrochemical-sensor system 100, in place of sensor module 103. As shown, the sensor module 1020 comprises a plurality of layers 1001 to 1010 made of suitable materials.

As shown, the sensor module 1020 has a bottom substrate layer comprising a holding substrate 1009 and a commercially available SPE 1010 that is prepared by specific functionalization process. Layers 1001 to 1009 may be collectively referred to herein as the microfluidic unit of the sensor module 1020. The microfluidic unit is configured to automate the electrochemical sensing steps. Each layer is described in detail below.

In some embodiments, the first substrate layer 1009 is made of PMMA with a thickness of 1-1.5 mm, is cut considering the dimensions of the SPE 1010.

In some embodiments, the second layer 1008 is made of PSA or other double sided adhesive tapes used as the substrate for the sample flow through the sensor module 1020. The areas around the SPE 1010 are also covered with PSA 1008 to provide a substrate for placing the nitrocellulose fibers 1007ii for delivering the sample to the SPE 1010.

In some embodiments, the low porous tissue 1007i covers the working electrode 1018 of the SPE 1010 to warrant a uniform distribution of the sample on the working electrode 1018. This configuration prevents the contact of the sample with the other two counter and reference electrodes of SPE 1010.

In some embodiments, the layer 1006 is made of PSA or other double-sided adhesives with hydrophilic contact angle to create the fluid channel through which the liquid sample flows from the inlet well 1016 to the sensing chamber 1013 and the vent 1011.

In some embodiments, the layer 1006 is made of PMMA or PSA with laser cut channel that forms the redox channel 1019. The redox channels (1015 and 1019) may have different designs such as curve, zigzag, or spiral shapes with the aim of facilitating the release of redox probes soaked in nitrocellulose fiber 1004 and placed within the redox channel 1019. In some embodiments, the downstream side of the redox channels 1015 and 1019 can be connected to the sensing chamber 1013 at different locations along the peripheral of this chamber. In some embodiments, the nitrocellulose fiber 1004 can be replaced with different medical grade absorbing pads with different porosity, hydrophilicity, liquid storage and release capacity, or electrical charges.

In some embodiments, the layer 1003 is made of PMMA or PSA with the thickness of 1-1.5 mm to shape the height of the redox channel 1015. The hydrophilicity of this layer 1003 can be adjusted to control the rate of liquid flow in the redox channel (1015 and 1019), important in controlling the delay needed for delivery of redox probes into the sensing chamber 1013 to stop immunoreactions on the working electrode 1018.

In some embodiments, the layer 1002 is made of PSA or PET which confines the fluid flow in the redox channels (1015 and 1019) while its hydrophilicity is tuned to control flow rate in this channel. In some embodiments, the layer 1001 is made of PMMA to seal the fluid flow in the microfluidic unit.

In some embodiments, the liquid sample dispensed in the inlet well 1012 can be different bodily fluids, for example nasal sample, serum, sweat, plasma, the whole blood, tear, or urine. In some embodiments, the nitrocellulose fiber 1004 is soaked in 30-70 mM solution of [Fe(CN)]63−/4− in PBS, dried, and assembled into the redox channel 1015.

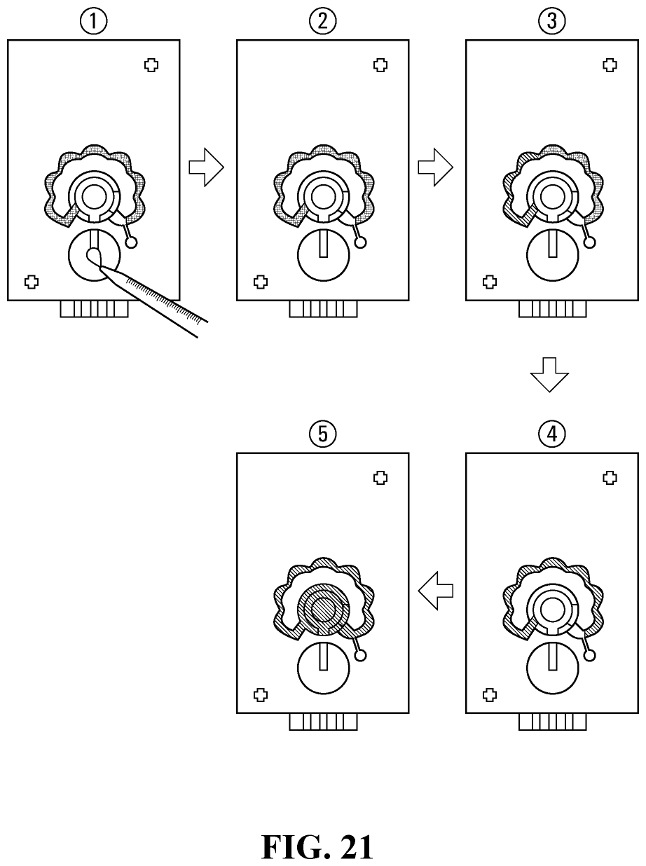

FIG. 21 illustrates the sequence of liquid flow in an assay of electrochemical immuno-biosensing automatically through which the self-powered microfluidic unit controls sequential delivery of the sample dispensed in the inlet well 1012 and the sensing reagents (redox probe soaked in the nitrocellulose fiber 1004) to the surface of the embedded SPE 1010. The sample containing the target biomarkers is dispensed into the inlet well 1012 (using a pipette or a custom-designed plastic dropper) with a defined volume (step 1 in FIG. 21). The microfluidic channel engraved inside the PSA layer 1006 guides the sample into the sensing chamber 1030 within 10-20 s, bring it over the embedded SPE 1010. To prevent damage to the capture molecules on the working electrode 1018 during filling up the sensing chamber 1013, a laser-cut low density tissue fiber 1007i with antifouling properties in the shape of the working electrode 1018 is placed in the PSA layer 1006 which helps to control the flow rate of the sample toward the working electrode 1018 (step 2 in FIG. 21). The immunoreaction (or any other interaction between the biomarker and the capture molecule) initiates immediately once the sample covers the working electrode 1018. Meanwhile, the redox-loaded nitrocellulose 1004 guides the remaining sample volume into the redox channel 1015 while the redox is gradually dissolved into the sample (step 3 in FIG. 21). The specific design of the redox channel 1015 provides the time appropriate for immunoreaction on the biosensor (5-60 min) and for better dissolving and mixing the redox reagent into the sample. The atmospheric pressure at the end of the redox channel at the vent 1017 and the outlet vent at the downstream channel of the sensing chamber 1011 contributes to controlling the pressure difference within the fluid network (step 4 in FIG. 21). The capillary delivery of the mixed redox sample into the sensing chamber 1013 ends the immunoreaction and displaces the sample with the redox-sample mixture through a combined diffusion and convection process. Once the sample-redox mixture fills the sensing chamber 1013, the electrochemical sensing recording is performed from the SPE 1010 (step 5 in FIG. 21).

In some embodiments, without any need for active fluid handling, the sample-to-result testing of sensor module 1020 offers immunoreaction within 5-60 minutes followed by the subsequent automatic washing, redox probe delivery, and electrochemical signal recording.

With reference to FIG. 20, in some embodiments, the diffusion level of one or multiple dried redox molecules (presoaked in nitrocellulose fiber 1004) into the sample and their concentrations and delayed delivery onto the sensing chamber 1013 are tunable, enabling electrochemical sensing from multiple working electrodes 1018 (like the electrode in FIG. 10 once embedded within the microfluidic unit) at different intervals (multiplex biosensing).

In some embodiments, multiple redox channels (1015/1019) with their own independent redox probe-soaked fibers 1004 can be arranged in series or parallel together, wherein one or multiple redox probes are delivered sequentially into the same or independent sensing chambers 1013 at different intervals to perform independent sensing from multiplex sensors (like the multiplex sensor in FIG. 13.

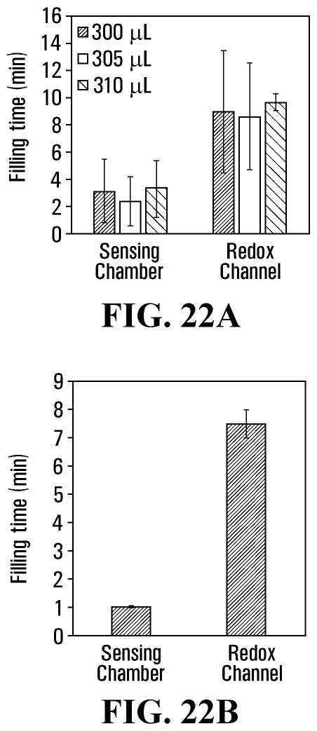

FIG. 22A illustrates the reproducibility of the microfluidic unit for filling time of the redox channel (1015/1019) and reproducible delivery of the sample-redox mixture to the sensing chamber 1013. This timing is dependent on viscosity of the sample, dimensions of the microfluidic unit, hydrophilicity levels of the fluidic layers and fibers, and volume of the sample. For example, three different sample volumes of 300 μL, 305 μL, and 310 μL PBS solution were tested in the microfluidic unit. While all these sample volumes successfully operated in the microfluidic unit by filling the sensing chamber 1013 and redox channel 1015/1019 in the intended intervals (15 min in this example), for the microfluidic unit tested with 310 μL PBS sample volume, we achieved 9.6±0.5 min filling time for the redox channel 2015/2019 with Relative Standard Deviation (RSD) of 6.66% (n=3) as opposed to the microfluidic units tested with 305 μL sample volume (RSD: 35.68%) (FIG. 22A). This demonstrates the highly reproducible microfluidic unit tested with 310 μL PBS sample volume. The performance of this microfluidic unit was also evaluated by testing it with human plasma samples, where the optimal sample volume for reaching high reproducibility was determined to be 350 uL with the sample leaving the redox channel 1013 in 8±1 min following the sample dispensing into the inlet well 1012 (FIG. 22B).

With reference to FIG. 20 (sensor unit 1020), in some embodiments, the SPE 1010 is coated with self-assembled L-cysteine functionalized Zinc Oxide/Reduced Graphene Oxide (ZnO/rGO) nanocomposite to provide an increased surface area and conductivity compared to bare SPEs and an enriched functionalized moieties for ultrasensitive detection of antigens, such as Nucleocapsid proteins, a known diagnostic biomarker of SARS-CoV-2 with a predictive performance to viral RNAs. This nano-construct benefits from ZnO nanoparticles with improved features of adsorbing of antibodies and stable covalent bonding with the thiolated self-assemble monolayer (L-cysteine), along with the enhanced receptor conjugation due to functional groups on the rGO, both important to make a sensitive and stable biosensor.

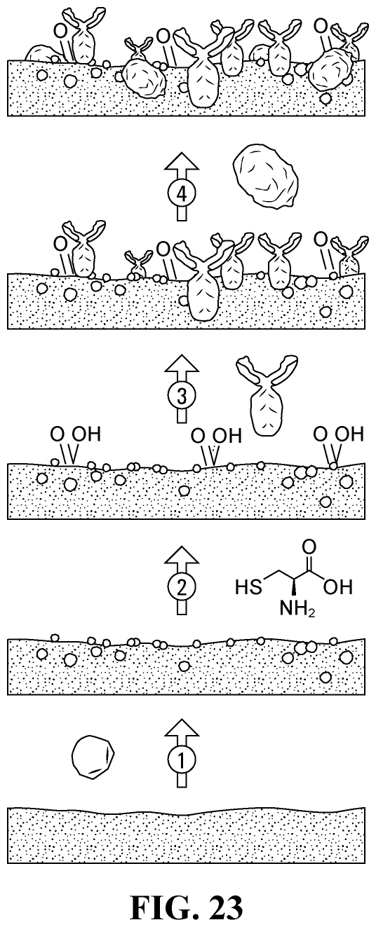

FIG. 23 illustrates the stepwise modification process of SPE 1010 for fabricating the ZnO/rGO-decorated N-protein sensor module. The bare SPE 1010 is first deposited with a nano-dispersion of ZnO/rGO in PBS (step 1), then carboxylated after incubation with L-Cysteine (step 2), immobilized with capture molecules (e.g., antibodies or aptamers) (step 3), coating with blocking reagents to prevent non-specific binding (e. g. bovine serum albumin (BSA)) (step 4), and ultimately interaction with the target analyte (biomarker) within the bodily fluid sample (step 5). The details of deposition of each layer could be similar to what reported for the sensor module 203.

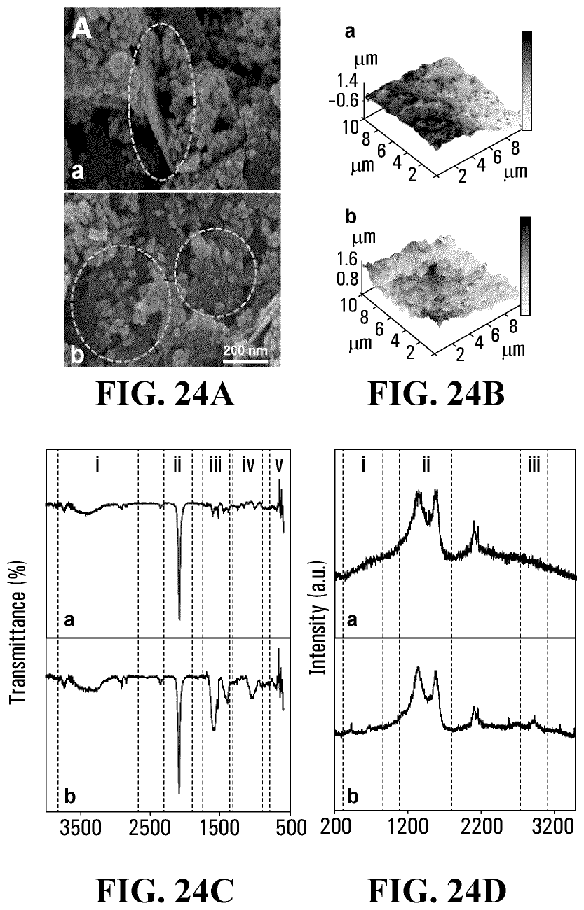

Physical and chemical characterizations have been performed on the coated layers in FIG. 23 to demonstrate proper deposition of the step-by-step deposition of these layers referred in FIG. 23. The presence of graphene nanosheets when added to the ZnO dispersion is shown in FIG. 24A, part a (step 1 in FIG. 23). It can be clearly seen that the graphene sheets limit the exposure of electron beams to the undelaying particles resulting in blur areas where the sheets are located, with the size and transparency as anticipated. The immobilization of the antibodies (step 4 in FIG. 23) was also assessed using the SEM images, where the surface change upon deposition of these antibodies are illustrated in FIG. 24A, part b. Surface topography analysis of ZnO/rGO and ZnO/rGO/Ab (antibody) was characterized using atomic force microscopy (AFM) tapping mode. As shown in FIG. 24B, the rGO nanosheets are uniformly distributed in the ZnO matrix and provide the smooth surface across the films. The average values of Root-Mean-Square (RMS) roughness (Rq) of ZnO/rGO and ZnO/rGO/Ab are 222 and 199 nm for the ZnO/rGO and ZnO/rGO/Ab, respectively. It can also be seen in FIG. 24B, parts a and b that the ZnO/rGO/Ab sample has smoother surface compared to the ZnO/rGO sample attributed to the fact that Ab filled the voids and made the smoother surface.

In reference to FIG. 24, ATR spectroscopy analysis has been conducted to perform functional analysis. The spectrum of ZnO/rGO shows a band at 1495 cm⁻¹ wavelength due to the skeletal vibration of the graphene, in addition to a broad absorption band at 3357 cm⁻¹ associated with the stretching vibrations of the hydroxyl groups of water molecules absorbed over the surface of ZnO/rGrO (FIG. 24C, part a). The existing C—OH and COO— bonds of 3430 cm⁻¹ and 2360 cm⁻¹, respectively, were found in the spectrum, which are assumably rooted in the porosity and hygroscopic nature of the graphene. The characteristic bonds at 1440, 1390, and 1230 cm⁻¹ were attributed to the C—H stretching, C—O stretching, and C—O vibration, correspondingly. Also, the peaks of S—O bonds were represented at 972 to 1010 cm⁻¹ wavelengths, in both spectrums, with higher intensity in the ZnO/rGO/L-Cysteine spectra (FIG. 24C, part b). Furthermore, the ATR spectrum was able to reveal the board O—H stretching vibration (3230-3554 cm⁻¹), C═O stretching vibration (1720-1750 cm⁻¹), and the associated Zn—O (597-704 cm⁻¹) functional groups. Through this analysis, the C═C peak from unoxidized sp2 C—C bonds was also observed at 1576-1620 cm⁻¹ wavelengths (FIG. 1C, part a), and with higher intensity in the ZnO/rG/L-Cysteine (FIG. 24C, part b). The spectrum of L-Cysteine was labelled as a (—NH) bending vibration at 1263 cm⁻¹, along with the peaks around 900 cm⁻¹, proving the existence of the —COOH functional group. The sharp peak of the C═O bond in both sample surfaces was also indicated at 2100 cm⁻¹.

Raman spectroscopy analysis has been performed to characterize surface-specific vibrational states of the ZnO/rGO and ZnO/rGO/L-Cysteine coatings (FIG. 24D, part a). The Raman spectrum illustrates two characteristic peaks situated at 1315 and 1595 cm-1, which are assigned as D and G peaks, respectively. The D peak is related to the breathing mode of k point phonons of A₁_{_{g}} symmetry that arises owing to local defects and disorders, particularly at the edges of graphene. The G peak, on the other hand, is ascribed to the E₂_{_{g}} phonon mode of sp2-hybridized carbon atoms presented within the graphene matrix. The Raman shift of ZnO was observed at 373 cm-1 generated from the second-order Raman spectrum arising from zone boundary phonons of the hexagonal ZnO. The peak that appeared at 445 cm-1 is attributed to the non-polar optical phonon E2 high-frequency mode of ZnO in the wurtzite structure from the C6v symmetry group. This confirmed that hexagonal wurtzite ZnO crystallites are formed on the graphene layer. The peak at 620 cm-1 corresponds to the E1 (LO) mode of the hexagonal ZnO, which is associated with oxygen deficiencies. The multiple-phono scattering is also observed at 1126 cm-1. The Raman spectrum of ZnO/rGO/L-Cysteine film (FIG. 24D, part b) also illustrates the similar characteristic peaks of ZnO, as well as G and D bands of ZnO/rGO. The intensity ratios of the D and G peaks (ID/IG) in ZnO/rGO and ZnO/rGO/L-Cysteine were determined to be 0.98 and 1.02, respectively. It is known that ZnO/rGO is a highly defective structure in which ID/IG decreases as an increasing defect density. Furthermore, the G peak in ZnO/rGO/L-Cysteine film is slightly shifted by 8 cm-1 compared to ZnO/rGO. The deviation of the G peak corresponds to the functionalization of organic molecules on the surfaces of carbon layers. This confirms that L-Cysteine molecules are functionalized on the surface of the rGO layer. Raman spectroscopy was also utilized to investigate the single-, bi-, and multilayer characteristics of rGO layers, with the 2D peak of the single-layer graphene sheet observed at 2878 cm-1.

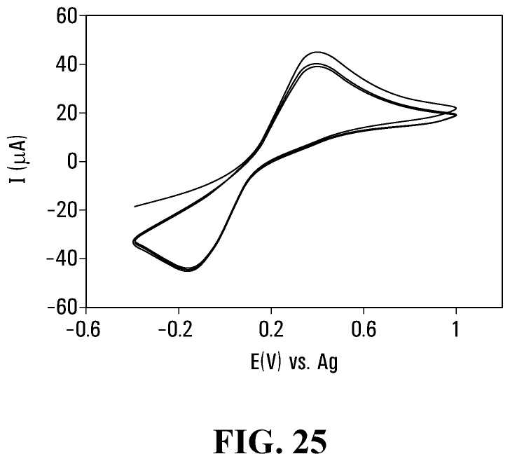

In FIG. 25, the stability of the coated nanomaterial composite ZnO/rGO on the surface of SPE 1010 has been evaluated by applying 10 cycles of the potential by the Cyclic Voltammetry (CV) technique. After coating the working electrode 1018 with the mixed nanomaterial dispersions (step 1 in FIG. 23), the coated working electrode 1018 was rinsed, dried, and underwent the CV measurements, within the potential window of −0.4 V to 1 V in a 2.5 mM [Fe(CN)6]3−/4− in PBS as the redox probe solution. Different ratios of ZnO to rGO dispersed in different solvents like PBS and NaCl were examined. Comparing the resulted of voltammograms for all different ratios and solvents showed that the combined ZnO and rGO with 2.5% (v/v) each, dispersed in PBS in the presence of the redox solution K3(Fe(CN)6)/K4(Fe(CN)6) has the highest stability with lowest peak current drop of 0.81% from the second oxidation peak in the CV cycles to the last peak (FIG. 25).

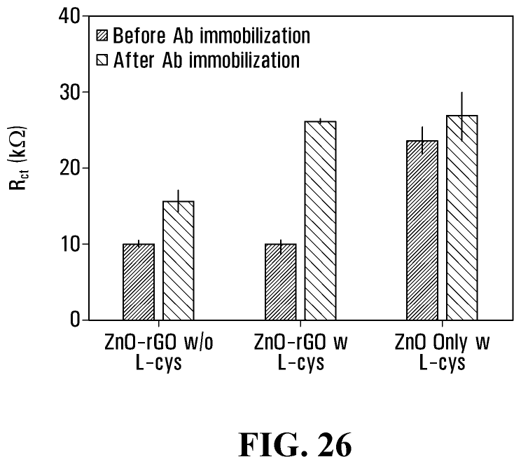

In FIG. 26, L-cysteine cross linker has been used on top of the ZnO/rGo coated working electrode 1018 (step 2 in FIG. 23) to use its carboxyl moieties for contributing to the level of capture molecules (e.g., antibodies here) immobilized on the working electrode 1018. The Ab immobilized ZnO/rGo coated electrodes in the absence of the crosslinker (L-cysteine) shows that the existence of the intrinsic carboxylic groups in the rGO sheets along with the electrical characteristic of ZnO for bioanalyte adsorption result in a rise in the charge transfer resistance (Rct) signals from 10.14±0.31 kΩ for Ab-free electrode to 15.60±1.37 kΩ for antibody deposited electrodes. This confirms the immobilization of the antibody bioreceptors on the surface (ΔRct=5.45 kΩ) in the absence of the crosslinker. However, once the ZnO/rGo coated electrodes were functionalized with 10 mM L-cysteine solution and formed a self-assembled monolayer containing carboxyl functional groups, the value of Rct signal changed from 9.60±0.88 kΩ for the pre-Ab immobilization step to 26.23±0.23 kΩ in Ab-immobilized working electrode 1018 (ΔRct=16.63 kΩ). This significant change in Rct indicates the effective role of L-cysteine in enhancing the surface potential for capturing the antibodies and hence increasing the sensitivity of the sensor module 1020.

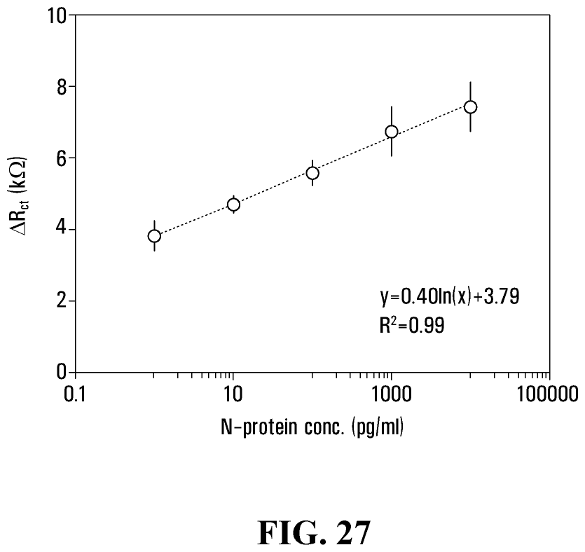

In FIG. 27, demonstrated is the high sensitivity of the sensor module 1020 prepared with the ZnO/rGO nanocomposite protocol (FIG. 24) for detection of Nucleocapsid (N—) proteins of SARS-CoV-2. Solution samples containing defined concentrations (0.5, 1, 10, 100, 1000, and 100,000 pg/mL) of N-proteins were prepared by a serial dilution of the stock N-protein into the PBS buffer. Optimizing the time involved for the completion of the antibody and antigen immunoreaction showed that the minimal interaction time needed is 15 min. EIS measurements were conducted for obtaining electron transfer resistance signals for recognizing the N-protein with increasing concentrations. The calibration curve of N-protein is shown in FIG. 27, demonstrating the overall sensitivity of 1280.73 ohms. mL/pg·mm2 and a limit of detection (LoD) of 0.02 pg/mL for the detection of N-protein. The linear detection range for the N-protein sensor module was found to be 1 pg/mL to 10 ng/ml, with the limit of quantification (LoQ) of 14.9 pg/mL.

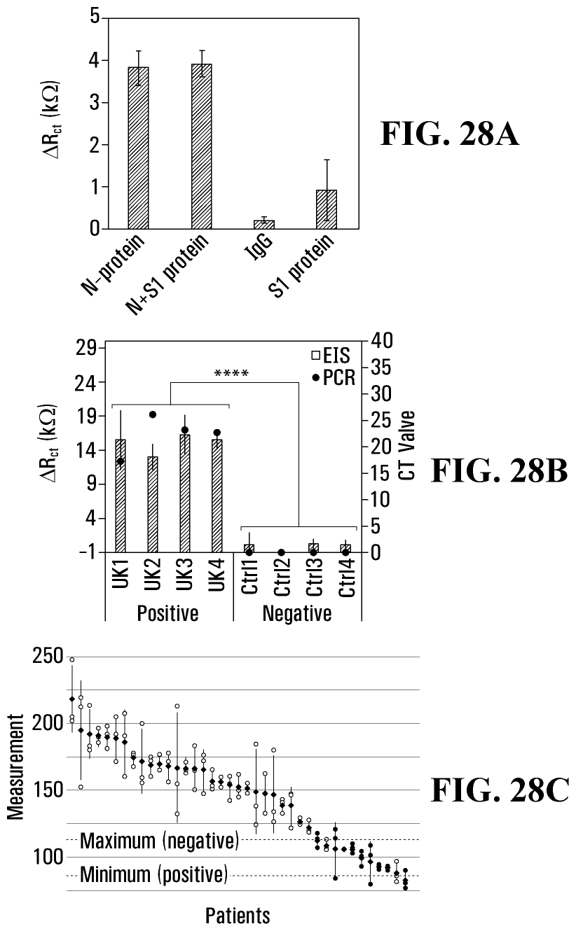

In FIG. 28A, demonstrated is the selective response of the ZnO/rGrO coated sensor module 1020 for detecting of N-proteins. The sensor module 1020 was first assessed through dispending of a solution containing Human Immunoglobulin G (IgG) and human SARS-CoV-2 Spiked (S1) proteins into the inlet well 1012. The rationale for this selection is related to the coexistence of IgG and S1 proteins (on the surface of the virus or freely available in the sample) along with N-proteins in clinical samples of infected individuals. The fabricated N-protein sensor unit 1020 was subject to different solutions, including only 1 pg/mL IgG, only 1 pg/mL S1 protein, and a solution containing 1 pg/mL of both S1 and N-proteins. The selectivity testing results show that the Rct signals for testing the sensor modules 1020 with the spiked samples containing IgG and S1 protein (0.21±0.07 and 0.91±0.72 kΩ, respectively) are significantly lower than the specific response of sensor modules 1020 tested with 1 pg/mL N-protein spiked samples (3.81±0.40 kΩ; P<0.05). Also, an insignificant difference comparing the Rct value of the sensor modules tested with N-protein spiked samples to those sensor modules tested with a combined S- and N-proteins spiked samples shows Rct=3.91±0.30 kΩ. This analysis confirms the high specificity of the N-protein sensor module to detecting N-protein analytes in the presence of protein interferences.

In FIGS. 28B and 28C, demonstrated is the clinical utility of the point-of-care N-protein sensor module 1020 for detecting of SARS-CoV-2 nucleocapsid protein in Nasopharyngeal samples positive or negative for SARS-CoV-2 as a sample-to-result test. The samples were initially collected in universal/viral transport media for clinical diagnosis of COVID-19 and frozen at −20° C. after testing. The first set of positive SARS-CoV-2 samples were identified as the Alpha variant of concern (B.1.1.7 lineage). The second set of positive SARS-CoV-2 were identified as wild-type samples. Also control samples include those without any viral infection and those with Flu A, Flu B, and RSV viral infections. The clinical sample testing was performed in a Biocontainment Level Class II laboratory, without any prior heat treatment, deactivation, or other sample preparation steps. For the first set of samples, four SARS-CoV-2 positive patients (with viral particles associated with the variant B.1.1.7) and four negative controls were tested with the N-protein sensor module 1020. Each positive and negative sample without any preparation were dispensed into the inlet well 1012 and incubated for 15 min before recording the EIS signals (1 min). The successful immunoreaction of N-proteins present in the NP swab samples of infected patients is confirmed via the significant difference (cut-off value=3 kΩ, P value<0.05) of their EIS signals compared to those obtained from the control group. The clinical samples were already tested using rRT-PCR, cT values of which are shown in FIG. 28B. The second set of clinical samples were tested similar to the first set, where the N-protein sensor module not only detected the positive sample with the clinical sensitivity of 95% and clinical specificity of 1005, it could also differentiate SARS-CoV-2 from other viral infections of Flu A, Flu B and RSV (FIG. 28C).

With reference to FIG. 20 (sensor module 1020), in some embodiments, the SPE 1010 is coated with nano-porous carbon electrodes modified with nitroso functional groups (e.g., hydroxylamine (NH₂OH)) and subject to cyclic voltammetry (CV) undergoing electrochemical oxidation to increase conductivity of the working electrode 1018. The drop casting of 1-ethyl-3-(3-dimethylaminopropyl) carbodiimide (EDC), N-hydroxysuccinimide (NHS) (EDC-NHS) crosslinker on the NH2OH functionalized working electrode 1018 provided binding site for the immobilization of capture molecules (antibodies or aptamers). Details for preparing the sensor system was presented in FIG. 17. In some embodiments, the redox probe can be 2.5-5 mM potassium ferrocyanide (K4Fe(CN)6) and potassium ferricyanide (K3Fe(CN)6) (1:1)).

The SPE 1010 (prepared with the protocol presented in FIG. 17) has been integrated the into the microfluidic unit of the sensor module 1020 (FIG. 20) to create a highly sensitive and quantitative GFAP point-of-care biosensor. This GFAP sensor module 1020, in one example, demonstrated its clinical value for point-of-care detection of patients with mild traumatic brain injuries (mTBI), stroke and spinal cord injuries (SCI). and concussion, something that has not been possible in any other point-of-care detection devices.

In some embodiments, different components of the microfluidic unit of sensor modules (203 or 1020) are assembled together in a modular microfluidic format. For example, the sample preparation compartment (including inlet wells 1012, filtration unit 1007i, ii in sensor module 1020) is one compartment made in one sub-microfluidic unit while the other compartments of redox channel 1015, nitrocellulose fiber 1004, sensing chamber 1013, and SPE 1010 is made in another sub-microfluidic unit. These two sub-microfluidic units are assembled easily together via different mechanisms (e.g. male/female connection), magnetic plugging, or a common underlying adhesive substrate.

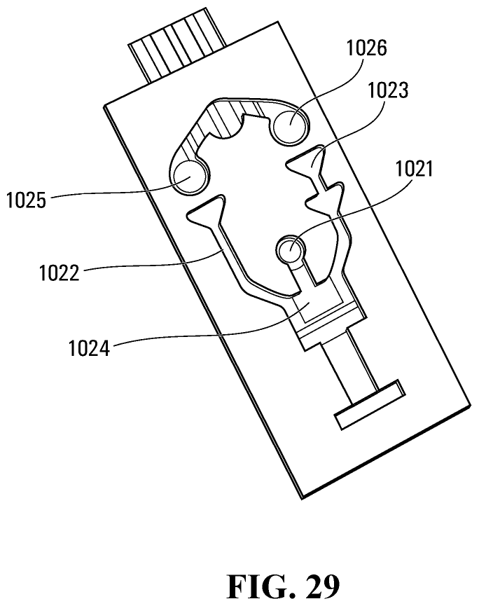

In some embodiments referred to the sensor modules 203 or 1020, the thick PMMA layers of the sensor modules 203 and 1020 are replaced with thin PSA or PET layers to significantly reduce the sample volume needed for making a self-power microfluidic unit (FIG. 29A). In some embodiments, the dimension of the inlet well 1021 is tunable to control the sample volume needed for each of functioning the side channels 1022 and 1023 and the sensing chamber 1024. In some embodiments, the washing side channel 1022 may be separate from the redox channel 1023. Therefore, by controlling the delay of liquid flow on each of these side channel and redox channels, the washing step can be automatically and separately done prior to the release of the redox probes from the redox channel 1022 to the sensing chamber. This sequential delivery of the washing buffer (presoaked in nitrocellulose fiber 1025) and redox probes (pre-soaked in nitrocellulose fiber 2016) is crucial for complex bodily fluid samples like the whole blood or serum.

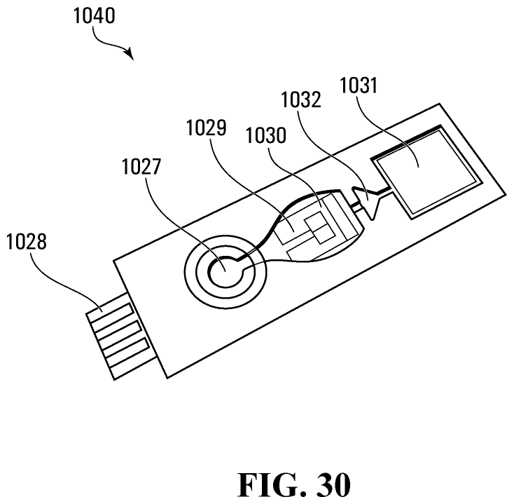

In some embodiments referred to the sensor modules 203 or 1020, the redox probe molecules may not be presoaked inside the microfluidic unit, but it would be added into the inlet well using a dropper after the completion of the immunoreaction on the working electrode 1018. FIG. 30 shows an alternative sensor module 1040, where the electrochemical biosensing is performed semi-automatically using a modification on the sensor module 1020. The workflow of sensing in this sensor module 1040 (FIG. 30) starts with dispensing the sample (10-15 uL) into the inlet well 1027 and flowing it using the capillary force of the microfluidic unit into the sensing chamber 1029 and on top of the working electrode 1030 of the screen-printed electrode 1028. Knowing that the trigger valve 1032 remained closed during the sample dispensing, the immunoreaction initiates immediately over the working electrode 1030. After about 10-30 min (depending upon the target biomarker), about 70 μL of the 1:1 ration washing buffer (DI) to redox probe will be dispensed into the inlet well 1027 using a small dropper (e.g., eye dropper) wherein this volume mixture activates and opens the trigger valve, flowing the initial sample out (drawn by the absorbing fiber 1031 at the vent), and replacing it with the fresh buffer-redox mixture. Upon filling the sensing chamber 1029 with this new buffer-redox solution, the electrochemical impedance signal (EIS) is recorded from the electrode 1028. This semi-automated point-of-care sensor module though is not fully automated, it can still be used with lay personnel as a point-of-care device. This sensor module 1040 is especially useful when the redox molecules cannot be easily soaked in the fiber or have a limited shelf life.

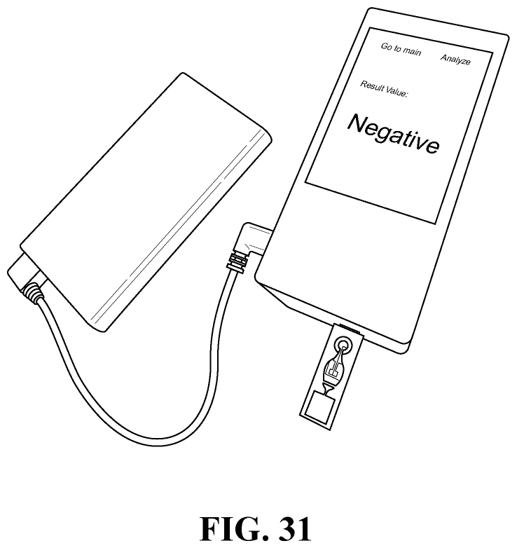

In some embodiments, the sensor modules 203, 1020 or 1040 are low cost (<$10) and can be easily linked to a hand-held reader (<$80; FIG. 31). While the sensor module is disposable, the reader can be used for a long period for electrochemical reading from the sensor modules, mainly for longitudinal monitoring of disease recovery or worsening.

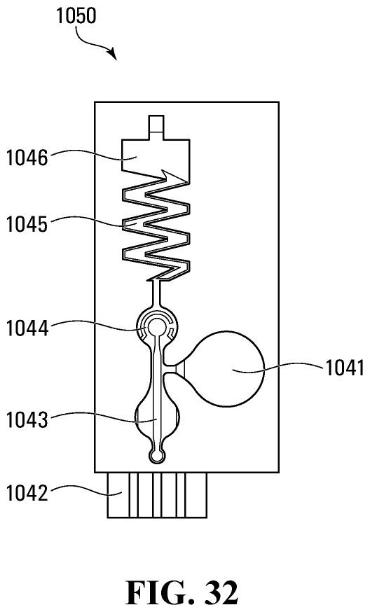

In some embodiments, with reference to the sensor modules 203 or 1020, the redox probe molecules may not be presoaked inside the fibers or added into the inlet well separately at specific timepoints. FIG. 32 shows an alternative sensor module 1050, where the electrochemical biosensing is performed automatically using a modification on the sensor module 1040. In chip 1050, the redox reagent is dispensed inside redox chamber 1043 and then dried before chip assembly. This enabled sequential delivery of the sample and redox solution on the sensing area (containing one or multiple working area) in a fully automated manner. The workflow of sensing in this sensor module 1050 (FIG. 32) starts with dispensing the sample (20-40 uL) into the inlet well 1041, with the bottom and top layers of the inlet well made from hydrophilic materials for contributing to the flow of the sample inside the chip via capillary force. The specific design of the inlet well 1041 enables full discharge of the total volume of injected sample, eliminating any risk of contamination from the inlet site. The whole microfluidic system is built on either a miniaturized version of one working electrode or multiplex electrode (electrochemical sensing strip), with the lead part 1042 being connected to the readout apparatus. The sample flows inside the redox chamber 1043 and fills it, where the redox concentrated solution (70 mM) has been drop-casted (1-2 uL) at the center of the chamber and dried at room temperature. The incoming sample first reaches the redox chamber and dissolves the dried redox, and then enters the sensing area 1044 on top of the biosensor where the immunoreaction occurs. The wicking fiber 1045 placed at downstream of the sensing chamber absorbs the incoming sample via its zig-zag channel controlling the incubation period of the bio-analytes with the biosensor. The immunoreaction occurs on the sensing area for 5-15 minutes depending on the bioanalyte of concern, while the pad slowly drags the redox solution from the redox chamber to the sensing area, simultaneously performing the washing step needed for removing specific bonding. The fiber 1046 placed at the end of the fluidic channels is directly connected to the wicking pad 1045, encapsulating the incoming sample up to the point of saturation. This provides the accurate period when the assay signal acquisition using electrochemical impedance spectroscopy (EIS), or voltammetry techniques is performed. This platform is a fully automated electrochemical point-of-care system, where all the process of redox preparation, washing, antibody/antigen interaction, and redox delivery on the electrochemical cell occurs autonomously. An increased shelf-life of the system due to the elimination of fiber-soaked redox is expected for this platform 1050. The entire chip is made of laser cut thin sheet layers without any need to expensive microfabrication process.

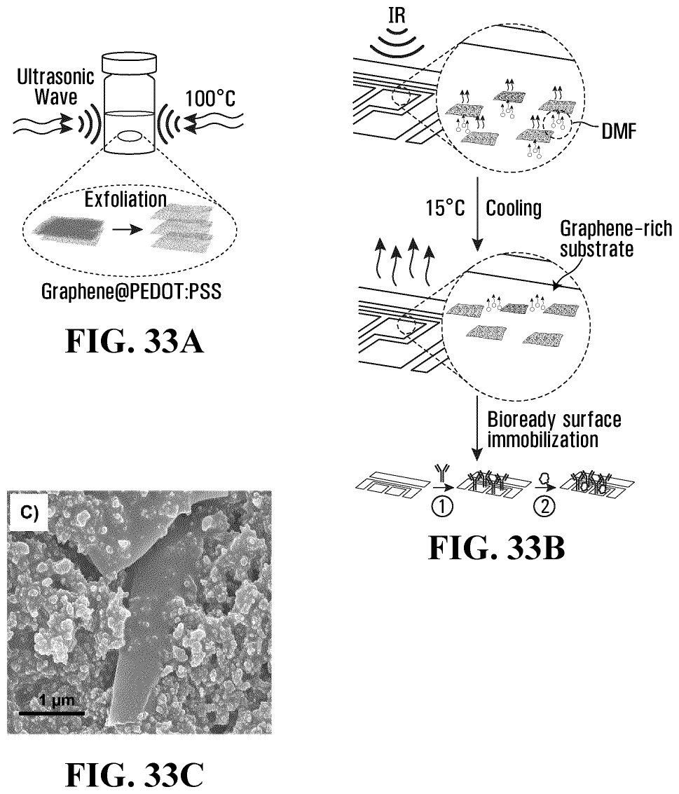

FIGS. 33A to 33C are schematic representations of a fabrication process of bio-ready sensing units containing simultaneous ultrasonic mixing and heating for graphene nanosheet delamination/exfoliation and infrared curing and cooling for sudden evaporation of solvents resulting in accumulation of the graphene nanosheets on the surface.

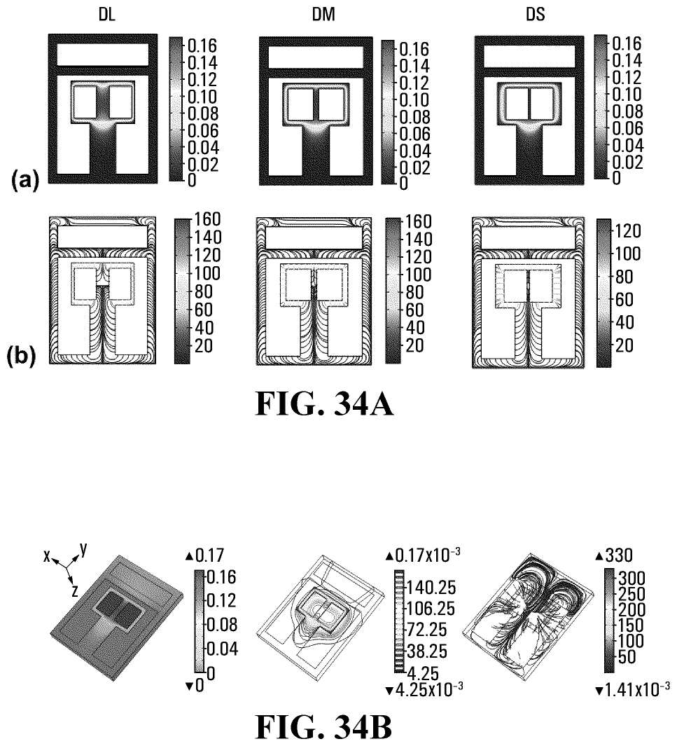

The design of sensor units 602 and 104 follow the principles of electrochemical sensor designs. The appropriate layout of the relative position of the reference, counter, and working electrodes with respect to one another was taken into consideration. For both working electrodes in the sensor unit 104, the counter electrodes cover peripheral areas of each of the working electrodes in a symmetric way and with identical interface resulting in equal signals acquired from each of the two working electrodes. The numerical simulations (FIGS. 34A and 34B) show identical current distribution between the working electrode 1 (WE1) and the working electrode (WE2) and the counter, confirming identical sensing performance. The reference electrode in sensor unit 104 is placed at a relatively larger distance from the counter electrodes, resulting in the elimination of the shielding effect. The design on the sensor unit 602 also represents similar interaction of each working electrode with the counter electrode in terms of accessible area for charge transfer, and placement of the reference electrodes at a relatively larger distance from the counter electrode. Commercial/conventional multi-working electrode systems only consider symmetry in their design, with minimal consideration of electrochemical sensor design principles in terms of placement of each electrode with respect to one another.

The dimensions of the sensor unit 104 in terms of relative distance of working electrodes are optimized with three different distances of large (DL, 750 μm), b) medium (DM, 300 μm), and c) small (DS, 125 μm) using numerical COMSOL simulations (FIGS. 34A and 34B). simulation The numerical enabled analysis of electrochemical/electrical response of electrodes for a vast range of dimensional and layout placements. The DM design results in equal electrical current distribution between each of the working electrodes and their corresponding counter electrodes, showing independent and comparable performance of each of the working electrodes. Potential and current distribution in two-dimensional layouts (FIG. 34A), and current streamlines in three-dimensional layouts (FIG. 34B) show the effectiveness of the layout design in yielding two functional sensors in one sensing unit enabling multiplex detection. A decrease or increase in the size of the electrodes up to 5 times of the original electrode size would not impose interference on cross-talk between the two working electrodes, enabling independent function of each working electrodes.

The process of intermixing the Graphene@PEDOT:PSS ink inside the carbon base ink is optimized. Synchronized ultrasonic mixing and heating at 100-110° C., which is less than the evaporation temperature of the DMF solvent, is used to delaminate/exfoliate the graphene nanosheets from each other. The process continued for 30-40 min in tandem with the addition of diacetone acrylamide (DAAM) binders, gradually added to the mixture. After mixing with the carbon ink as described in process 500, infrared (IR) curing is applied for 5 min at 150° C. (as the evaporation temperature of the DMF) and immediately cooled down to external fan temperature (˜15° C.). The process uniquely enables accumulation of graphene nanosheets on the surface of electrodes providing proper access to functional groups of the graphene nanosheets.

### Clauses

Additional aspects are described by the following clauses:

Clause 1. A hand-held point-of-care apparatus comprising an electrochemical-sensor system, a self-powered capillary microfluidic unit, and a hand-held and low-cost reader.
Clause 2. The apparatus of clause 1 wherein the electrochemical sensor is decorated with highly conductive nanomaterial and crosslinker composites for quantitative, digital, rapid, and stable detection of target biomolecules and bioparticles in different bodily fluids.
Clause 3. The apparatus of clause 1 wherein the electrochemical sensor is created on a conductive ink with its intrinsic functional groups enabling zero-step functionalization of the electrodes for reproducible immobilization of capture molecules and for sensitive and rapid detection of biomolecules and bioparticles.
Clause 4. The apparatus of any one of clauses 1-3 wherein a multi-electrode configuration, printed by the conductive inks or prepared using nanomaterial composites, enables simultaneous detection and quantification of multiple bodily fluid markers in different bodily fluids including blood, serum, plasma, nasal, sweat, saliva, tear and urine.
Clause 5. The apparatus of clause 1 wherein the self-powered capillary microfluidic chip is a multilayer microfluidic unit configured for sample preparation and target extraction/purification customized for different bodily fluids and biomarkers while automating the steps of electrochemical biosensing all within the same fluidic unit. The microfluidic unit comprising:
i) an inlet reservoir to collect the bodily fluids from dispensers or swabs and filter the target biomarkers,
ii) a network of surface engineered channel networks and embedded fibers, drop-casted concentrated solutions, for preparation of the sample and conducting it to only the working electrodes of the biosensors,
iii) a network of pre-soak fibers, engineered side channels, and downstream solution preparation chambers to automate the process of electrochemical biosensing (incubation, washing, redox probe preparation, and sensing) customized for one or multiple biomarkers.
   Clause 6. The apparatus of any one of clauses 1-5, wherein the electrochemical sensing module is easily assembled and embedded into the microfluidic unit using a compatible double-sided tape without any leakage or complexity of aligning the layers.
   Clause 7. The apparatus of claims 1-6 capable of automatic and point-of-care detecting multiple biomarkers within one single self-powered microfluidic devices.
   Clause 8. The apparatus of any one of clauses 1-6 that enabled point-of-care detection of proteins with a sensitivity and limit of detection of 100-1000 times higher than the commercial ELISA kits but equivalent to single ELISA molecule assays (SIMOA) technology.
   Clause 9. The apparatus of any one of clauses 1-6 that created point-of-care GFAP protein biosensing for rapid and accurate diagnosis and prognosis of patients with brain injuries, stroke and spinal cord injuries.
   Clause 10. The apparatus of any one of clauses 1-6 created point-of-care detection devices detecting SARS-CoV-2 antigens for rapid diagnosis of SARS-CoV-2 in clinical samples.
   Clause 11. The apparatus of any one of clauses 1-10 wherein their reader module comprising an electronic reader with embedded electronic components, a processor, a memory, data storage, and/or analog/digital circuitry. The reader is as low-cost as $50-100, useful for point-of-care and on-site detection applications.
   Clause 12. A system for detecting one or more target biomolecules or bioparticles in a sample point-of-care device is presented. The system has a reader module for detecting the target biomolecule/bioparticle based on a set of input electrical signals and a sensor module comprising a sensor unit and optionally a self-powered microfluidic unit. The sensor unit has one or more electrodes (decorated with novel nonmaterial composites or made by conductive nanomaterial-enriched inks) that are functionalized with a capture molecule (e.g. antibody or aptamer) that corresponds to the target biomolecule/bioparticle, and is configured to receive a sample solution directly or via the microfluidic unit and provides the input electrical signals to the reader module based on a samples solution. The microfluidic unit is fully capillary-based and self-powered, and has one or more microchannel layers having microfluidic channels, filters, and fibers for receiving therein a hydrophilic material and filtering, transporting, extracting, and purifying the sample solution towards a biosensing chamber configured to fluidly communicate with the sensor. There are several side fluid channels connected to the main sample transport channel that take care of automating the entire process of sample preparation and sensing, including filtration, purification, extraction, dilution, delayed delivery of redox probes, fluorescent dyes or washing buffer to the biosensing chamber, enabling fully automation of sample-to-result test. Based on the input electrical signals, the reader module can detect the presence and concentration of the multiple target biomarkers.

Numerous modifications and variations of the present disclosure are possible in light of the above teachings. It is therefore to be understood that within the scope of the appended claims, the disclosure may be practised otherwise than as specifically described herein.

1. A sensor unit configured for use in an electrochemical-sensor system having a reader module and a microfluidic unit, the sensor unit comprising:
a distal portion configured to connect with the microfluidic unit for receiving a sample;
a proximal portion configured to connect with the reader module for measuring electrochemical properties of the sample;
a substrate extending from the distal portion to the proximal portion and enabling fluid to flow thereon;
a first working electrode distributed on the substrate and extending from the distal portion to the proximal portion, such that the first working electrode has a sampling end in the distal portion and a connecting end in the proximal portion;
a second working electrode distributed on the substrate and extending from the distal portion to the proximal portion, such that the second working electrode has a sampling end in the distal portion and a connecting end in the proximal portion; and
a control electrode distributed on the substrate and extending from the distal portion to the proximal portion, such that the control electrode has a connecting end in the proximal portion and wraps around the first working electrode and the second working electrode in the distal portion;
wherein the sampling end of the first working electrode and the sampling end of the second working electrode are separated from each other by a defined distance in a range of 125 to 750 μm;
wherein the first working electrode and the second working electrode have a combined surface area, and the control electrode has a defined surface area, such that a ratio of the combined surface area of the working electrodes to the defined surface area of the control electrode is in a range of 0.2 to 1.25.

2. The sensor unit of claim 1, wherein the ratio of the combined surface area of the working electrodes to the defined surface area of the control electrode is in a range of 0.25 to 1.25.

3. The sensor unit of claim 1, wherein the first working electrode and the second working electrode have identical surface area.

4. The sensor unit of claim 1, wherein the first working electrode and the second working electrode are separated from each other by the defined distance between the proximal portion and the distal portion.

5. The sensor unit of claim 4, wherein the defined distance between the first working electrode and the second working electrode is constant between the proximal portion and the distal portion.

6. The sensor unit of claim 1, further comprising:
a reference electrode distributed on the substrate and extending from the distal portion to the proximal portion.

7. The sensor unit of claim 1, wherein the first working electrode and the second working electrode each comprise a nanostructured-sensing surface having a plurality of capture areas.

8. The sensor unit of claim 7, wherein the nanostructured-sensing surface comprises 10-20% w/w poly(3,4-ethylenedioxythiophene) polystyrene sulfonate (PEDOT:PSS)/Graphene nanocomposite and 80-90% w/w Graphite.

9. A combination, comprising:
a microfluidic unit; and
a sensor unit in accordance with claim 1.

10. The combination of claim 9, wherein the sensor unit and the microfluidic unit are both part of a single integrated component which is a sensor module.

11. The combination of claim 10, wherein the sensor module comprises:
an inlet reservoir in the form of a hole for receiving the sample;
a side redox route wherein a presoaked redox reagents mixes with the sample; and
a biosensing chamber wherein an antibody-antigen interaction occurs.

12. The combination of claim 9, wherein the combination is an electrochemical-sensor system and further comprises a reader module.

13. A sensor unit configured for use in an electrochemical-sensor system having a reader module and a microfluidic unit, the sensor unit comprising:
a distal portion configured to connect with the microfluidic unit for receiving a sample;
a proximal portion configured to connect with the reader module for measuring electrochemical properties of the sample;
a substrate extending from the distal portion to the proximal portion and enabling fluid to flow thereon; and
a plurality of electrodes distributed on the substrate and extending from the distal portion to the proximal portion;
wherein each electrode comprises a nanostructured-sensing surface having a plurality of capture areas, and wherein the nanostructured-sensing surface comprises 10-20% w/w poly(3,4-ethylenedioxythiophene) polystyrene sulfonate (PEDOT:PSS)/Graphene nanocomposite and 80-90% w/w Graphite.

14. The sensor unit of claim 13, wherein the nanostructured-sensing surface has an average thickness of 7±2 μm.

15. The sensor unit of claim 14, wherein the average thickness is 7±1 μm.

16. A combination, comprising:
a microfluidic unit; and
a sensor unit in accordance with claim 13.

17. The combination of claim 16, wherein the sensor unit and the microfluidic unit are both parts of a single integrated component which is a sensor module.

18. The combination of claim 17, wherein the sensor module comprises:
an inlet reservoir in the form of a hole for receiving the sample;
a side redox route wherein a presoaked redox reagents mixes with the sample; and
a biosensing chamber wherein an antibody-antigen interaction occurs.

19. The combination of claim 16, wherein the combination is an electrochemical-sensor system and further comprises a reader module.

20. A method, comprising:
applying conductive ink onto a substrate such that, upon the conductive ink drying, an electrode having a nanostructured-sensing surface is formed on the substrate;
wherein the conductive ink comprises 0.2-0.3 mg/mL PEDOT:PSS mixed with 1-2 mg/mL electrochemically exfoliated graphene.

21. The method of claim 20, wherein the applying of the conductive ink is executed such that the nanostructured-sensing surface that is formed has an average thickness of 7±2 μm.

22. The method of claim 21, wherein the average thickness is 7±1 μm.

23. A conductive ink comprising 0.2-0.3 mg/mL PEDOT:PSS mixed with 1-2 mg/mL electrochemically exfoliated graphene.

24. A microfluidic unit, comprising:
a microchannel comprising a sample-receiving section configured to receive a sample, an electrodes-interface section configured to supply the sample to electrodes of a sensor unit, and a gap between the sample-receiving section and the electrodes-interface section configured to slow down movement of the sample and thereby delay the supplying of the sample to the electrodes.

25. The microfluidic unit of claim 24, wherein the gap in the microchannel has a length of 2 to 5 mm.

26. The microfluidic unit of claim 24, wherein the sample-receiving section of the microchannel comprises a non-linear shape to increase a path of travel for the sample.

27. The microfluidic unit of claim 26, wherein the non-linear shape comprises a spiral shape.

28. The microfluidic unit of claim 26, wherein the non-linear shape comprises a zigzag shape.

29. The microfluidic unit of claim 24, wherein the microchannel has a channel width of about 100 μm to 1 mm.

30. The microfluidic unit of claim 24, wherein the electrodes-interface section of the microchannel comprises a redox probe.

31. The microfluidic unit of claim 24, comprising a chamber with a redox probe.

32. A combination, comprising:
a sensor unit; and
a microfluidic unit in accordance with claim 24.

33. The combination of claim 32, wherein the sensor unit and the microfluidic unit are both part of a single integrated component which is a sensor module.

34. The combination of claim 32, wherein the sensor module comprises:
an inlet reservoir in the form of a hole for receiving the sample;
a side redox route wherein a presoaked redox reagents mixes with the sample; and
a biosensing chamber wherein an antibody-antigen interaction occurs.

35. The combination of claim 32, wherein the combination is an electrochemical-sensor system and further comprises a reader module.
